SEQUENCE LISTING (22 sequences)

SEQ ID NO: 1 - protein, 824 aa, Homo sapiens
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 2 - RNA, 21 nt, synthetic construct
  source 1..21: mol_type = other RNA
gaagttcatgagccttggccc

SEQ ID NO: 3 - RNA, 20 nt, synthetic construct
  source 1..20: mol_type = other RNA
ggtttgtggactatacttgt

SEQ ID NO: 4 - RNA, 21 nt, synthetic construct
  source 1..21: mol_type = other RNA
agccttggcccactgtagatt

SEQ ID NO: 5 - RNA, 20 nt, synthetic construct
  source 1..20: mol_type = other RNA
tcatgagccttggcccactg

SEQ ID NO: 6 - RNA, 20 nt, synthetic construct
  source 1..20: mol_type = other RNA
aagttcatgagccttggccc

SEQ ID NO: 7 - RNA, 22 nt, synthetic construct
  source 1..22: mol_type = other RNA
ggtggtttgtggactatacttg

SEQ ID NO: 8 - RNA, 20 nt, synthetic construct
  source 1..20: mol_type = other RNA
ttgtggactatacttgtgta

SEQ ID NO: 9 - RNA, 20 nt, synthetic construct
  source 1..20: mol_type = other RNA
tagaggcagtgcttgggaag

SEQ ID NO: 10 - RNA, 21 nt, synthetic construct
  source 1..21: mol_type = other RNA
gaggcagtgcttgggaaggtc

SEQ ID NO: 11 - RNA, 20 nt, synthetic construct
  source 1..20: mol_type = other RNA
gtccagccaacactgccttg

SEQ ID NO: 12 - RNA, 20 nt, synthetic construct
  source 1..20: mol_type = other RNA
caagagcagatttattttgt

SEQ ID NO: 13 - RNA, 20 nt, synthetic construct
  source 1..20: mol_type = other RNA
tgatcttaccatatactcct

SEQ ID NO: 14 - RNA, 21 nt, synthetic construct
  source 1..21: mol_type = other RNA
ttacaatgatcttaccatata

SEQ ID NO: 15 - RNA, 20 nt, synthetic construct
  source 1..20: mol_type = other RNA
aatataacaatcctgggagt

SEQ ID NO: 16 - RNA, 24 nt, synthetic construct
  source 1..24: mol_type = other RNA
aatataacaatcctgggagtgggg

SEQ ID NO: 17 - RNA, 23 nt, synthetic construct
  source 1..23: mol_type = other RNA
attttaccttttccgacacatat

SEQ ID NO: 18 - RNA, 21 nt, synthetic construct
  source 1..21: mol_type = other RNA
gagacattttaccttttccga

SEQ ID NO: 19 - RNA, 20 nt, synthetic construct
  source 1..20: mol_type = other RNA
cgtagtcatttctatttata

SEQ ID NO: 20 - RNA, 21 nt, synthetic construct
  source 1..21: mol_type = other RNA
tcatcgtagtcatttctattt

SEQ ID NO: 21 - RNA, 22 nt, synthetic construct
  source 1..22: mol_type = other RNA
caacgatgcttacgtggcatat

SEQ ID NO: 22 - protein, 821 aa, Mus musculus
MSLWGQPLQASPPLAVRQPPTASSGPSTSPPAGATLNRLPEPLLRRLSESLDRAPEGRGWRQLAELAGSRGRLRLSGLDLEQCSLKVLEPEGSPSLCLLKLMGEKGCTVTELSDFLQALEHTEVLPLLNPPGLKITVNPESKAVLAGQFVKLCCRATGHPFVQYQWFKMNKEIPYGNSSELVFNTVHVKDAGFYVCRVNNSSTFEFSQWSQLDVCDVAEVTDSFQGSMDGISESRLQICVEPRSQRLVPGSMLLLQCVAIGSPMPHYQWFKDESPLTHETKKHYTVPYVDIEHEGTYWCHVYNDRDSQDSKKAEVTIDELNNLGHPDNKEQTGQPLAKDKVALLIGNMSYWEHPKLKAPLVDVYELTNLLRQLDFKVVSLLDLTEYEMCNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGRQALEIRSSLSEKRALTDPVQGAPCSAEALVRNLQWAKAHELPESMCLKFQCGVHIQLGFAAEFSNVMIIYTSIVHKPPEIIMCDAYVTDFPLDLDIDPKHANKGTPEETGSYLVSKDLPKHCLYTRLSSLQKLKEHLIFTVCLSYQYSGLEDTVEEKQEVNVGKPLIAKLDMHRGLGRKTCFQACRMPDEPYHSSTSTSAGAGHFHSSQDSFHDVYHSHLGNADSGMPPDRCHCSRTPHTFISNYPPHHYCQFGRSNVPVETTDEMPFSFSDRLMISEN

(19) United States
(12) Patent Application Publication
(10) Pub. No.: US 2026/0258425 A1
(43) Pub. Date: Sep. 3, 2026

(54) MALT1 TUMOR INHIBITOR, PHARMACEUTICAL COMPOSITION CONTAINING THE SAME, AND APPLICATIONS THEREOF

(71) Applicant: XZYP (BEIJING) BIOTECH CO., LTD, Beijing (CN)

(72) Inventor: Daojun CHANG, Beijing (CN)

(73) Assignee: XZYP (BEIJING) BIOTECH CO., LTD, Beijing (CN)

(21) Appl. No.: 19/489,247

(22) PCT Filed: Jun. 3, 2024
(86) PCT No.: PCT/CN2024/097119

§ 371 Date: Dec. 1, 2025

Related U.S. Application Data
(60) Provisional application No. 63/470,373, filed on Jun. 1, 2023.

(51) Int. Cl. C12N 15/113; A61K 31/7115; A61K 31/712; A61K 31/7125; A61K 39/00; A61K 45/06; A61P 35/00; C07K 16/28; G01N 33/68

(52) U.S. Cl. CPC C12N 15/1137; A61K 31/7115; A61K 31/712; A61K 31/7125; A61K 45/06; A61P 35/00; C07K 16/2818; C07K 16/2827; G01N 33/6845; A61K 2039/505; C12N 2310/11; C12N 2310/14; C12N 2310/31; C12N 2310/32; C12N 2310/33; C12Y 304/22; G01N 2333/4706; G01N 2333/96466

(57) ABSTRACT

The present invention provides a MALT1 tumor inhibitor. This inhibitor can inhibit the expression of the MALT1 gene or inactivate the function of the MALT1 protein. It is selected from agents that can affect the expression of the death domain of the MALT1 protein, agents that knockout the death domain, agents that alter the structure of the death domain, or agents that degrade the death domain. The MALT1 tumor inhibitor can effectively treat various solid tumors and can also treat tumors that are untreatable with immune checkpoint inhibitors. In addition, when used in combination with immune checkpoint inhibitors, it can exert a synergistic effect, and the therapeutic effect is significantly enhanced.

### TECHNICAL FIELD

The present application relates to the field of pharmaceutical biotechnology, and specifically discloses MALT1 tumor therapeutic agents, pharmaceutical compositions and other products containing the same, as well as applications of such products in the field of tumor treatment.

### RELATED ART

MALT1 is a cysteine-dependent aspartate-specific protease with high homology to members of the Para-caspase family, and plays a key role in the activation of the NF-κB signaling pathway.

The structure of the MALT1 protein mainly consists of three parts: a Para-caspase domain, a death domain, and an immunoglobulin-like domain.

MALT1 can cleave a variety of substrates, including CYLD, A20, and RelB, all of which are negative regulators of the NF-κB signaling pathway. Therefore, in addition to serving as a scaffold to participate in the formation of complexes in the NF-κB signaling pathway, MALT1 can also exert proteolytic enzyme activity to participate in and activate NF-κB. In addition, MALT1 can induce the growth of lymphocytes and stimulate the transcription of the cytokine interleukin-2 to participate in immune responses. Thus, MALT1 inhibitors can effectively inhibit the intracellular activation of MALT1, which is of great significance for immune regulation involving MALT1 and the treatment of lymphoma.

Studies have shown that the MALT1 protein is proven to be associated with hematological malignancies. Research on MALT1 includes:

Cornell University disclosed aryltriazole compounds (2-chloro-N-[4-[5-(3,4-dichlorophenyl)-3-(2-methoxyethoxy)-1H-1,2,4-triazol-1-yl]phenyl]acetamide, hereinafter referred to as MI-2) that inhibit MALT1 activity by binding to the Para-caspase fragment in Patent Document WO2014/074815. Subsequently, Cornell University further disclosed a series of derivatives of MI-2 in Patent Document WO2017/040304, whose structures are shown in FIG. 1. These compounds all covalently bind to the Para-caspase fragment of MALT1 and can significantly inhibit the development of ABC-DLBCL tumors in in vitro and in vivo experiments.

Hatcher J M et al. disclosed a series of aryltriazole compounds (WO2014/074815). These compounds carry a reactive chloromethyl warhead and are proposed to covalently bind to the catalytic C464 in the para-aspase domain of MALT1, see FIG. 1.

Helmholtz Zentrum München disclosed a series of phenothiazine compounds and their derivatives for use as MALT1 inhibitors in Patent Documents WO2013/017637, WO2014/086478, and WO2014/207067, including mepazine, promazine, and thioridazine (see FIG. 2), showing that such MALT1 inhibitors have good biochemical and cellular activities.

Novartis disclosed a series of pyrazolopyrimidine derivatives as MALT1 inhibitors in Patent Documents WO2015/181747 and WO2017/081641, see FIG. 3.

Janssen disclosed a series of trifluoromethylpyrazoles as MALT1 inhibitors in Patent Documents WO2019/243965, US2019/0381012, and US2019/0381019, see FIG. 4.

However, the aforementioned MALT1 inhibitors all target the typical active center region of MALT1, namely the Para-caspase fragment; and there are few research reports on such MALT1 inhibitors in solid tumors.

In addition, GE Healthcare disclosed MALT1 inhibitors targeting the CARMA2,3-BCL10-MALT1 complex in Patent Document CN111770759A. Caspase recruitment domain-containing membrane-associated guanylate kinase protein (CARMA) belongs to the membrane-associated guanylate kinase family. There are three members of the CARMA protein family: CARMA1, CARMA2, and CARMA3. Among them, CARMA1 is mainly expressed in lymphoid tissues, including the spleen, thymus, and peripheral blood; CARMA2 is mainly expressed in the skin and mucous membranes, with the level in the skin being 5 times higher than that in other tissues and organs; CARMA3 is widely expressed in various organs and tissues such as the heart, kidneys, and liver. Therefore, the CARMA2,3-BCL10-MALT1 complex disclosed in Patent Document CN111770759A is only disclosed to function in immune cells.

### SUMMARY OF INVENTION

Through experiments, the present application found that inhibiting the activity of the binding region between BCL10 and MALT1 can disrupt the formation process of the CARMA3-BCL10-MALT1 complex (hereinafter referred to as the CBM complex). Based on this, the present application first discovered an effector region for tumor treatment (hereinafter referred to as the MALT1 tumor effector region) in the structure of the MALT1 protein, which plays an important role in the protein-protein interaction between MALT1 and BCL-10. Unlike the catalytic active center of the Para-caspase of MALT1, the MALT1 tumor effector region is located in the death domain of MALT1.

Experimental verification has confirmed that the MALT1 tumor effector region is a target with tumor therapeutic effects. Therefore, the MALT1 tumor effector region, the base sequence expressing the death domain, and the protein-protein interaction site between MALT1 and BCL-10 can all serve as targets or sites with beneficial effects on tumor treatment. Intervening in the MALT1 tumor effector region, the base sequence expressing the death domain, and the protein-protein interaction site between MALT1 and BCL-10 can achieve the effect of treating tumors.

Accordingly, the present application provides antisense oligonucleotides targeting the MALT1 tumor effector region, wherein the nucleotide sequence of the antisense oligonucleotide is as shown in any one of SEQ ID No. 2-6 and 8-12.

Based on the MALT1 tumor effector region, the present application also proposes an inhibitor (hereinafter referred to as the MALT1 tumor inhibitor) that can effectively treat tumors, especially solid tumors, by acting on the MALT1 tumor effector region.

The MALT1 tumor effector region is located far from the catalytic active center of the Para-caspase of MALT1. Therefore, the MALT1 tumor inhibitor has the characteristics of non-competitive inhibition with MALT1 enzyme activity inhibitors, and can be used in combination with existing inhibitors targeting the MALT1 Para-caspase active center to achieve better therapeutic effects.

Accordingly, the present application provides a pharmaceutical composition including the MALT1 tumor inhibitor and a pharmaceutically acceptable carrier.

More importantly, unlike existing MALT1 inhibitors that only exert effects in hematological tumors, the MALT1 tumor inhibitor of the present application also exerts good therapeutic effects in solid tumors, which has never been reported before in the prior art.

Therefore, the present application provides applications of the MALT1 tumor inhibitor and the pharmaceutical composition in the preparation of drugs for treating tumors.

In the present application, the MALT1 tumor inhibitor that inhibits the activity of the MALT1 tumor effector region can be used to treat various cancers, including hematological cancers and solid cancers, especially including but not limited to malignant tumors occurring in the lungs, pancreas, liver, digestive tract, reproductive system and other parts, such as Hodgkin's lymphoma, non-Hodgkin's lymphoma, lung cancer, breast cancer (including triple-negative breast cancer), melanoma, ovarian cancer, gastric cancer, colon cancer, rectal cancer, liver cancer, pancreatic cancer, head and neck malignant tumors, urinary system malignant tumors, endometrial cancer, cervical cancer, osteosarcoma, chondrosarcoma, Ewing's sarcoma, thyroid cancer, hepatoblastoma, nephroblastoma, etc.

### BRIEF DESCRIPTION OF DRAWINGS

FIG. 1 shows the structural formulas of aryltriazole compounds that covalently bind to the catalytic C464 in the para-aspase domain of MALT1;

FIG. 2 shows the structural formulas of phenothiazine compounds and their derivatives as MALT1 inhibitors;

FIG. 3 shows the structural formulas of pyrazolopyrimidine derivatives as MALT1 inhibitors;

FIG. 4 shows the structural formulas of trifluoromethylpyrazoles as MALT1 inhibitors;

FIG. 5 shows the Western blot results of the antisense oligonucleotide sequences of E0771 cells 48 hours after transfection with Lipofectamine™ 3000 transfection reagent, where 5 μL of Lipofectamine™ 3000 reagent was added to each well of a 12-well plate;

FIG. 6 shows the tumor growth curves corresponding to mutant strains at different sites of the MALT1 death domain;

FIG. 7 shows the Western Blot results of 11 cell strains after co-immunoprecipitation;

FIG. 8 shows the tumor cell survival rates in 11 cell strains before and after the addition of macrophages;

FIG. 9 shows the corresponding relationship between mutant strains at different sites of the MALT1 death domain and the number of macrophages;

FIG. 10 shows the visualized results of cell counts obtained by direct photography under a microscope;

FIG. 11 shows the single-parameter histogram results of flow cytometry;

FIG. 12 shows the corresponding relationships between mutant strains at different sites of the MALT1 death domain and macrophage ratio, CD206 intensity (M2 marker), and MHCI intensity (M1 marker);

FIG. 13 shows the tumor immune cell infiltration in each mouse detected by flow cytometry;

FIG. 14 shows the corresponding relationship between target-mutated cell strains and tumor volume;

FIG. 15 shows the flow cytometry experimental results verifying the conversion of macrophages from M2-type to M1-type macrophages;

FIG. 16 shows that VEC, M-WT, M-WT-C83A, M-WT-V87R, M-WT-L88D, and M-WT-E104R (marked from top to bottom on the right side of FIG. 16) were respectively complemented in B16F10-MALT1-KD cells (melanoma cells) to obtain corresponding cell lines, and then 1×10⁵ corresponding cells were subcutaneously injected into the subcutaneous tissue near the groin of wild-type mice, showing the tumor size on day 18;

FIG. 17 shows that VEC, M-WT, M-WT-C83A, M-WT-V87R, M-WT-L88D, and M-WT-E104R (marked from top to bottom on the right side of FIG. 17) were respectively complemented in E0771-MALT1-KD cells (breast cancer cells) to obtain corresponding cell lines, and then 1×10⁵ corresponding cells were orthotopically injected into the fat pad of wild-type mice, showing the tumor size on day 19;

FIG. 18 shows that the antisense oligonucleotide prodrug can significantly inhibit the further development of cancer cells, as verified in breast cancer cell E0771;

FIG. 19 shows that B16F10-CTRL, B16F10-MALT1-KD1, and B16F10-MALT1-KD2 were co-cultured with CD8+ T cells respectively, and luciferase activity was measured after 24 hours to evaluate the survival rate of tumor cells (A of FIG. 19), and the tumor volume change curve in the animal model (B of FIG. 19);

FIG. 20 shows that MC38-CTRL, MC38-MALT1-KD1, and MC38-MALT1-KD2 were co-cultured with CD8+ T cells respectively, and luciferase activity was measured after 24 hours to evaluate the survival rate of tumor cells (A of FIG. 20), and the tumor volume change curve in the animal model (B of FIG. 20);

FIG. 21 shows the verification results of the survival rate of mouse breast cancer E0771 cells (A and B of FIG. 21);

FIG. 22 shows the verification results of the survival rate of mouse melanoma B16F10 cells (A and B of FIG. 22).

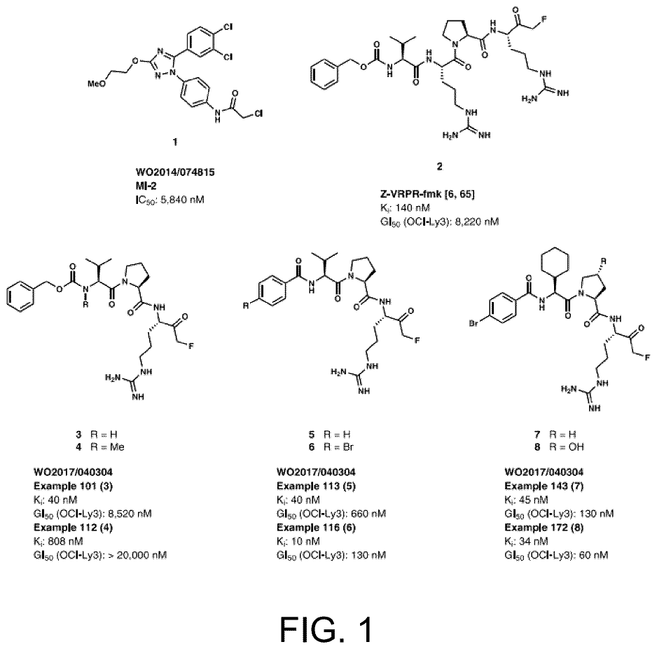

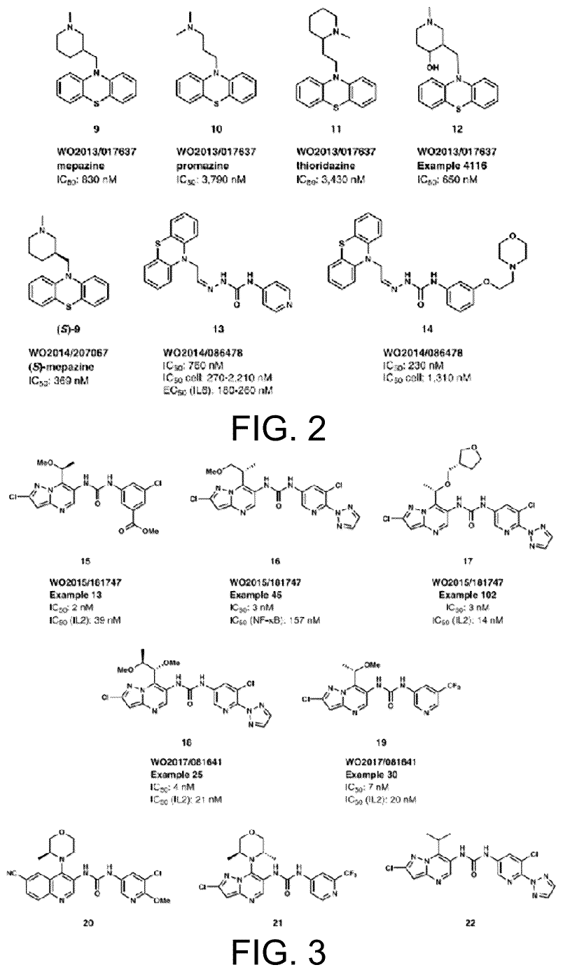

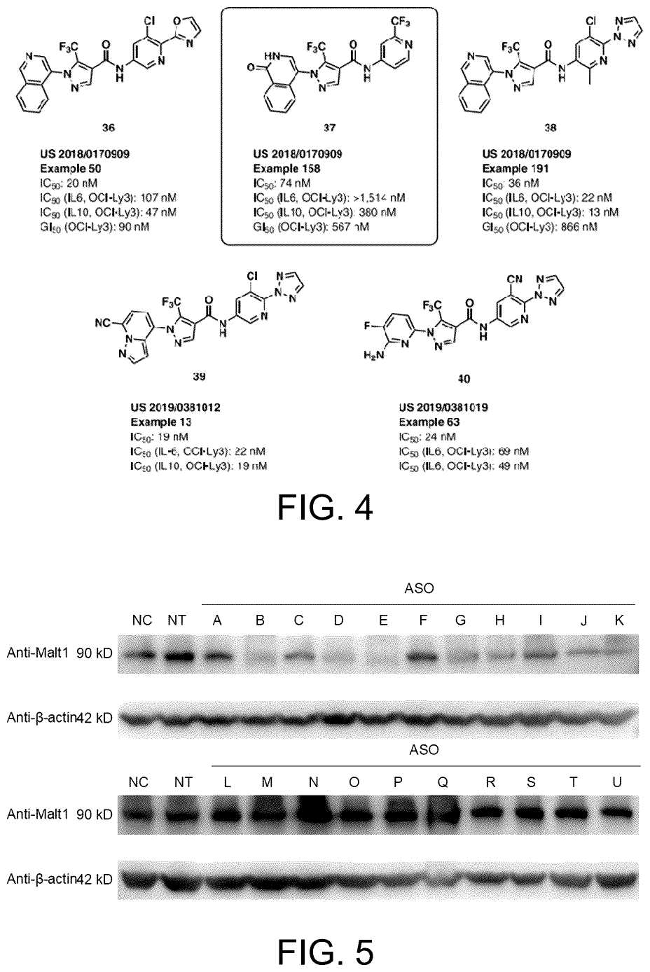

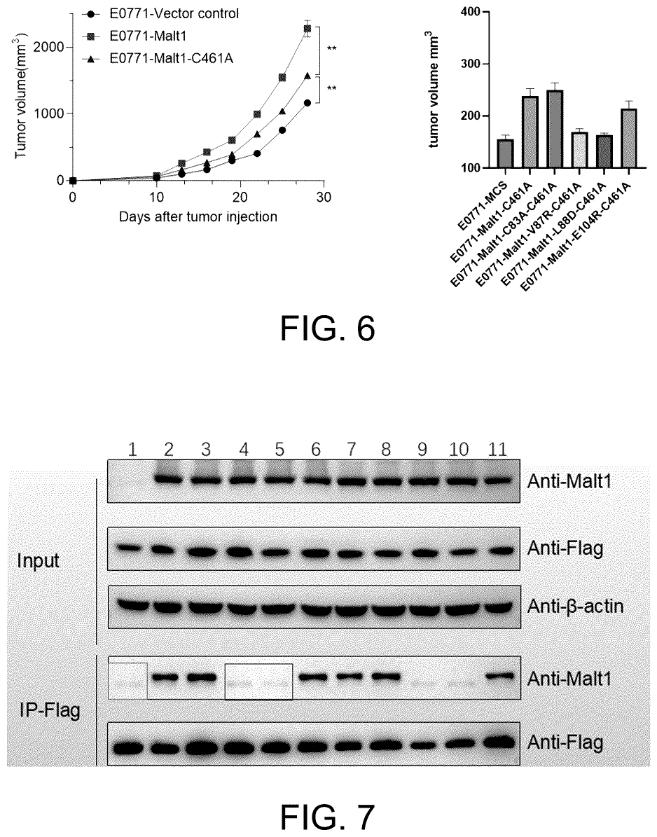

### DESCRIPTION OF EMBODIMENTS

### Terms and Definitions

“NCBI Gene ID” and “NCBI NP” are provided by the National Center for Biotechnology Information (NCBI), see the website at https://www.ncbi.nlm.nih.gov/.

The “MALT1 gene” (NCBI Gene ID: 10892) encodes a caspase-like protease that functions in BCL10-induced NF-κB activation. This protein is a component of the CARMA3-BCL10-MALT1 (CBM) signalosome, which can trigger NF-κB signaling and lymphocyte activation after antigen-receptor stimulation. MALT1 may refer to human MALT1, including its naturally occurring variants, molecules, and alleles. MALT1 may also refer to MALT1 of mammals such as mice, rats, rabbits, dogs, cats, cattle, horses, and pigs.

The “CARMA3-BCL10-MALT1 signalosome complex” refers to a trimolecular protein complex composed of caspase recruitment domain and membrane-associated guanylate kinase-like domain-containing protein 3 (i.e., CARMA3), B-cell lymphoma/leukemia 10 (BCL10), and mucosa-associated lymphoid tissue lymphoma translocation protein 1, i.e., composed of CARMA3, BCL10, and MALT1. After antigen-receptor ligation and signal cascade, CARMA3 is released from its autoinhibitory conformation and binds to BCL10 and MALT1 to form a functional CARMA2,3-BCL10-MALT1 complex.

“Inhibiting the activity of the CARMA2,3-BCL10-MALT1 complex” refers to the formation, function, or expression level of the CARMA2,3-BCL10-MALT1 complex. In some embodiments, the activity is the formation level of the CARMA2,3-BCL10-MALT1 complex. In some embodiments, to inhibit the activity of the CARMA2,3-BCL10-MALT1 complex, a subject needs to be administered a MALT1 tumor inhibitor that inhibits the activity of the CARMA2,3-BCL10-MALT1 complex.

In some embodiments, the MALT1 tumor inhibitor can inhibit the activity of the CARMA2,3-BCL10-MALT1 complex by at least 10%, at least 20%, at least 30%, at least 40%, at least 50%, at least 60%, at least 70%, at least 80%, at least 90%, at least 99% or more compared to the control level. The control level may be, for example, the activity of the CARMA2,3-BCL10-MALT1 complex before administration of the MALT1 tumor inhibitor, or the activity of the CARMA2,3-BCL10-MALT1 complex in cells not treated (e.g., contacted) with the MALT1 tumor inhibitor.

In some embodiments, the MALT1 tumor inhibitor inhibits the activity of the CARMA2,3-BCL10-MALT1 complex in T cells or in tumor cells. In some embodiments, the tumor cells are tumor-infiltrating tumor cells. In some embodiments, as measured by the amount of intact complex in the cell, the MALT1 tumor inhibitor reduces the amount of intact CARMA2,3-BCL10-MALT1 complex by at least 10%, at least 20%, at least 30%, at least 40%, at least 50%, at least 60%, at least 70%, at least 80%, at least 90%, at least 99% or more compared to the control level.

In some embodiments, the MALT1 tumor inhibitor of the present application inhibits the formation of the complex. The MALT1 tumor inhibitor can inhibit the formation of the complex by binding to and inhibiting binding sites contained in the complex required for higher-order assembly (such as the MALT1 binding site on BCL10). In some embodiments, the MALT1 tumor inhibitor can inhibit the release of CARMA3 from its autoinhibitory conformation. In some embodiments, the MALT1 tumor inhibitor inhibits upstream factors required for the formation of the CARMA2,3-BCL10-MALT1 complex.

In some embodiments, the MALT1 tumor inhibitor can reduce the expression level of at least one component of the CARMA2,3-BCL10-MALT1 complex by at least 10%, at least 20%, at least 30%, at least 40%, at least 50%, at least 60%, at least 70%, at least 80%, at least 90%, at least 99% or more compared to the control level. The control level may be, for example, the expression level of at least one component (such as CARMA3, BCL10, or MALT1) before administration of the MALT1 tumor inhibitor. Those skilled in the art can use, for example, co-immunoprecipitation or sucrose gradient analysis to evaluate the integrity of the complex to determine whether the MALT1 tumor inhibitor effectively inhibits the formation of the CARMA2,3-BCL10-MALT1 complex. For a given component, those skilled in the art can perform PCR-based analysis or Western blot to evaluate mRNA levels or protein levels, respectively, to determine whether the level of at least one component in the complex is reduced.

In some embodiments, the activity is the function of the CARMA2,3-BCL10-MALT1 complex. In some embodiments, the MALT1 tumor inhibitor inhibits the CARMA2,3-BCL10-MALT1 complex from activating its downstream targets (such as NF-κB nuclear translocation and activation).

In some embodiments, the MALT1 tumor inhibitor reduces the Para-caspase activity of MALT1 by at least 10%, at least 20%, at least 30%, at least 40%, at least 50%, at least 60%, at least 70%, at least 80%, at least 90%, at least 99% or more compared to the control level. The control level may be, for example, the Para-caspase activity of MALT1 before administration of the MALT1 inhibitor.

In some embodiments, the MALT1 tumor inhibitor inhibits the interaction with MALT1 substrates or the activation of MALT1 substrates by at least 10%, at least 20%, at least 30%, at least 40%, at least 50%, at least 60%, at least 70%, at least 80%, at least 90%, at least 99% or more. The control level may be, for example, the interaction with MALT1 substrates or the activation of MALT1 substrates before administration of the MALT1 tumor inhibitor.

In some embodiments, the MALT1 tumor inhibitor reduces the cleavage of MALT1 substrates by at least 10%, at least 20%, at least 30%, at least 40%, at least 50%, at least 60%, at least 70%, at least 80%, at least 90%, at least 99% or more. The control level may be, for example, the cleavage of MALT1 substrates before administration of the MALT1 tumor inhibitor.

In some embodiments, the MALT1 tumor inhibitor reduces the monoubiquitination of MALT1 by at least 10%, at least 20%, at least 30%, at least 40%, at least 50%, at least 60%, at least 70%, at least 80%, at least 90%, at least 99% or more. The control level may be, for example, the monoubiquitination of MALT1 before administration of the MALT1 tumor inhibitor. Those skilled in the art will be able to use standard assays (such as immunofluorescence to detect nuclear NF-κB) to determine whether the MALT1 tumor inhibitor effectively inhibits the activation of downstream targets.

In some embodiments, the activity is the expression level of the CARMA2,3-BCL10-MALT1 complex.

In some embodiments, the MALT1 tumor inhibitor reduces the expression level of the CARMA2,3-BCL10-MALT1 complex by at least 10%, at least 20%, at least 30%, at least 40%, at least 50%, at least 60%, at least 70%, at least 80%, at least 90%, at least 99% or more compared to the control level. The control level may be, for example, the level of the CARMA2,3-BCL10-MALT1 complex before administration of the MALT1 tumor inhibitor.

In some embodiments, the MALT1 tumor inhibitor reduces the expression level of at least one gene selected from the CARMA3 gene, BCL10 gene, or MALT1 gene by 10%, at least 20%, at least 30%, at least 40%, at least 50%, at least 60%, at least 70%, at least 80%, at least 90%, at least 99% or more compared to the control level. The control level may be, for example, the expression level of the CARMA3 gene, BCL10 gene, or MALT1 gene before administration of the MALT1 tumor inhibitor. Methods known in the art can be used to determine whether the MALT1 tumor inhibitor reduces the expression level of a gene (e.g., by PCR-based assays) or a gene product (e.g., by Western blot).

A “tumor antigen” refers to an antigen differentially expressed by cancer cells and thus can be exploited to target cancer cells. Tumor antigens are antigens that can potentially stimulate a significant tumor-specific immune response. Although not necessarily expressed, some of these antigens are encoded by normal cells. These antigens can be characterized as: antigens that are usually silent (i.e., not expressed) in normal cells; antigens expressed only at specific stages of differentiation; and transiently expressed antigens (such as embryonic and fetal antigens). Other tumor antigens are encoded by mutated cellular genes, such as oncogenes (e.g., activated ras oncogenes), suppressor genes (e.g., mutated p53), and fusion proteins produced by internal deletions or chromosomal translocations. Other tumor antigens can also be encoded by viral genes, such as genes carried on RNA and DNA tumor viruses. Many tumor antigens have been identified for various solid tumors: MAGE1, MAGE2, and MAGE3, determined by immunity; MART-1/Melan-A, gp100, carcinoembryonic antigen (CEA), HER2, mucins (i.e., MUC-1), prostate-specific antigen (PSA), and prostatic acid phosphatase (PAP). In addition, viral proteins such as some proteins encoded by hepatitis B virus (HBV), Epstein-Barr virus (EBV), and human papillomavirus (HPV) have been shown to be important in the development of hepatocellular carcinoma, lymphoma, and cervical cancer, respectively.

“Engineered” and its equivalents refer to one or more artificially designed alterations of nucleic acids. In some embodiments, engineering may refer to the alteration, addition, and/or deletion of genes.

An “engineered cell” may refer to a cell with added, deleted, and/or altered genes.

A “cell” or “engineered cell” and equivalents may refer to cells of human or non-human animal origin.

A “polypeptide” refers to a polymer of amino acids.

“Protein” and “polypeptide” are used interchangeably in the present application. Peptides are relatively short polypeptides, usually about 2 to 60 amino acids in length. Polypeptides used in the present application typically include amino acids such as the 20 most common L-amino acids found in proteins. However, other amino acids and/or amino acid analogs known in the art can be used. One or more amino acids in a polypeptide can be modified by, for example, adding chemical entities (such as carbohydrate groups; phosphate groups; fatty acid groups; linkers for conjugation, functionalization, etc.). A polypeptide having a non-polypeptide moiety covalently or non-covalently associated therewith is still considered a “polypeptide.” Exemplary modifications include glycosylation and palmitoylation. Polypeptides can be purified from natural sources, produced using recombinant DNA technology, or synthesized by chemical means (such as conventional solid-phase peptide synthesis), etc.

A “polypeptide sequence” or “amino acid sequence” may refer to the polypeptide material itself and/or sequence information that biochemically characterizes the polypeptide (i.e., consecutive letters or three-letter codes used as abbreviations for amino acid names). Unless otherwise indicated, polypeptide sequences presented in the present application are presented in the N-terminal to C-terminal direction.

In some embodiments, a nucleic acid encoding a polypeptide (e.g., an inhibitory polypeptide) described in the present application is contained in a vector.

In some embodiments, a nucleic acid sequence encoding a given polypeptide or any module thereof described in the present application is operably linked to a vector.

A “vector” refers to a nucleic acid construct designed for delivery to a host cell or for transfer between different host cells. Vectors used in the present application can be viral or non-viral.

A “vector” encompasses any genetic element that is capable of replication when associated with suitable control elements and can transfer gene sequences to cells. Vectors may include, but are not limited to, cloning vectors, expression vectors, plasmids, phages, transposons, cosmids, artificial chromosomes, viruses, virions, etc.

An “expression vector” refers to a vector that directs the expression of RNA or a polypeptide from a sequence linked to transcriptional regulatory sequences on the vector. The expressed sequence is usually, but not necessarily, heterologous to the cell. An expression vector may contain additional elements, for example, an expression vector may have two replication systems, allowing it to be maintained in two organisms, such as for expression in human cells and for cloning and amplification in a prokaryotic host.

“Expression” refers to cellular processes involved in producing RNA and proteins, and optionally secreting proteins, including, if applicable, but not limited to, transcription, transcript processing, translation, and protein folding, modification, and processing.

An “expression product” includes RNA transcribed from a gene and a polypeptide obtained by translation of mRNA transcribed from a gene.

A “gene” refers to a nucleic acid sequence that is transcribed (DNA) into RNA in vitro or in vivo when operably linked to appropriate regulatory sequences. A gene may or may not include regions before and after the coding region, such as 5′ untranslated (5′ UTR) sequences or “leader” sequences, and 3′ UTR sequences or “trailer” sequences, as well as intervening sequences (introns) between individual coding segments (exons).

The “tumor effector region” in the present invention refers to a continuous sequence of the MALT-1 death domain, approximately from amino acid residue 64 to 114 at the N-terminus of MALT-1. This region is a key structure that binds to BCL-10 to form the CARMA3-BCL10-MALT1 complex, thereby activating downstream pathways. By altering the structure of this tumor effector region, the function of the MALT-1 protein can be inactivated, and tumors can be inhibited or treated.

The “MALT1 tumor inhibitor” as used herein refers to an agent that inhibits the expression of the MALT-1 tumor effector region at the nucleic acid level or inactivates its activity at the protein level. In some embodiments, the MALT1 tumor inhibitor can be a nucleic acid that inhibits the expression of the MALT-1 death domain: siRNA, shRNA, ASO, double-stranded nucleic acid. In other embodiments, the MALT1 tumor inhibitor can be an agent that mutates the binding site of the tumor effector region/death domain to BCL10, and the MALT1 tumor inhibitor can also be an agent that performs homologous recombination on the gene fragment encoding the binding site of the tumor effector region/death domain to BCL10. In other embodiments, the MALT1 tumor inhibitor can also be an agent that degrades the MALT-1 protein, such as PROTAC, molecular glue.

The “MALT1 enzymatic activity inhibitor” as used herein refers to an activity inhibitor targeting the Para-caspase domain. It is known that the constitutive activity of MALT1 protease drives the survival and proliferation of various lymphoid malignancies. Therefore, MALT1 enzymatic activity inhibitors can effectively inhibit the survival and growth of tumors. In some embodiments, the MALT1 enzymatic activity inhibitor is a small molecule inhibitor having structural formulas 1-47 herein.

An “antisense oligonucleotide” refers to a class of oligonucleotides that inhibit gene expression by sequence-specifically hybridizing to a continuous sequence on the target gene DNA or mRNA. It is a molecular drug regulated at the gene level, with a length between 5 and 40 nucleotides. The antisense oligonucleotides of the present invention are single-stranded.

A “nucleotide” is the structural unit of an oligonucleotide. Nucleotides such as DNA and RNA nucleotides include a ribose moiety, a nucleobase moiety, and one or more phosphate groups.

A “modified nucleoside” is a bond other than a nucleic acid diester bond that covalently couples two nucleosides together. The oligonucleotides of the present invention may contain modified internucleoside linkages. In some embodiments, modified internucleoside linkages increase the nuclease resistance of the oligonucleotide compared to phosphodiester bonds. Modified internucleoside linkages are particularly useful for stabilizing oligonucleotides for in vivo use and can protect against nuclease cleavage both within DNA or RNA nucleoside regions of the oligonucleotide (e.g., within the gap region of a gapmer oligonucleotide) and within modified nucleoside regions.

“Homology” refers to the degree of similarity between the nucleotide sequences of two nucleic acid molecules or the amino acid sequences of two protein molecules in molecular evolutionary research.

A “pharmaceutical composition” refers to an active agent combined with a pharmaceutically acceptable carrier (e.g., a carrier commonly used in the pharmaceutical industry).

An “agent” may be in the form of a salt (e.g., a salt with an organic or inorganic acid). Examples of suitable acids for forming such acid addition salts include trifluoroacetic acid, hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, isethionic acid, vinylsulfonic acid, p-toluenesulfonic acid, naphthalenesulfonic acid, sulfanilic acid, camphorsulfonic acid, china acid, mandelic acid, o-methylmandelic acid, hydro-benzenesulfonic acid, picric acid, adipic acid, d-o-tolyltartaric acid, tartronic acid, (o-, m-, p-) methylbenzoic acid, naphthylaminesulfonic acid, and other inorganic or carboxylic acids known in the art. Salts are prepared by contacting the free base form with a sufficient amount of the desired acid to produce the salt in the conventional manner.

“Pharmaceutically acceptable” refers to compounds, materials, compositions, and/or dosage forms that, within the scope of sound medical judgment, are suitable for contact with the tissues of humans and animals without excessive toxicity, irritation, allergic response, or other problems or complications, and are commensurate with a reasonable benefit/risk ratio.

In some embodiments, the pharmaceutically acceptable carrier may be a carrier other than water.

In some embodiments, the pharmaceutically acceptable carrier may be an artificial carrier or an engineered carrier, for example, a carrier in which the active ingredient is not found naturally.

“Administration” refers to placing a therapeutic agent (e.g., an agent) or pharmaceutical composition disclosed in the present application in a subject by a method or route that results in at least partial delivery of the agent to the desired site. Pharmaceutical compositions containing the agents disclosed in the present application can be administered by any appropriate route that produces an effective treatment in the subject.

In some embodiments, administration is systemic administration.

“Systemic administration” refers to a route of administering an agent into the circulatory system of a subject. In some embodiments, administration is local administration.

“Local administration” refers to administering an agent to the site of action (e.g., a tumor). Local administration may be desirable to avoid adverse side effects caused by systemic administration of the therapeutic agent or pharmaceutical composition.

“Combination administration” refers to delivering two (or more) different treatments to a subject during the course of the subject's affliction with a disorder, for example, after the subject is diagnosed with a disorder or disease (e.g., a tumor) and before the disorder is cured or eliminated or the treatment is stopped for other reasons.

A “unit dosage form” refers to a dosage suitable for a single administration. For example, a unit dosage form may be an amount of a therapeutic agent arranged in a delivery device (e.g., a syringe or intravenous drip bag).

In some embodiments, a unit dosage form is administered as a single dose.

In some embodiments, more than one unit dosage form may be administered simultaneously.

An “effective amount” refers to the amount of an agent required to alleviate at least one symptom of a tumor (e.g., headache). Thus, the term “therapeutically effective amount” refers to the amount of an agent sufficient to provide a specific anti-cancer effect when administered to a typical subject. An effective amount as used in the present application also includes an amount of an agent sufficient to delay the development of tumor symptoms, alter the course of tumor symptoms (e.g., but not limited to slowing tumor progression), or reverse tumor symptoms. Therefore, specifying an exact “effective amount” is usually impractical. However, an appropriate “effective amount” can be determined by one of ordinary skill in the art using only routine experimentation for any given situation. Effective amount, toxicity, and therapeutic efficacy can be evaluated by standard pharmaceutical procedures in cell cultures or experimental animals. Dosages may vary depending on the dosage form employed and the route of administration used. The dose ratio between toxic and therapeutic effects is the therapeutic index and can be expressed as the ratio LD50/ED50. Compositions and methods that exhibit high therapeutic indices are preferred.

A “therapeutically effective dose” can be initially estimated from cell culture assays. In addition, dosages can be formulated in animal models to achieve a circulating plasma concentration range that includes the IC50 as determined in cell culture or in an appropriate animal model. Levels in plasma can be measured by, for example, high-performance liquid chromatography. The effect of any particular dosage can be monitored by suitable bioassays (e.g., non-invasive imaging), etc. The dosage can be determined by the physician and adjusted as necessary to suit the observed therapeutic effect.

“Statistically significant” or “significantly” refers to statistical significance, and generally refers to a difference of 2 standard deviations (2SD) or more. Except in the operating examples or where otherwise indicated, all numbers expressing quantities of ingredients or reaction conditions used in the present application are to be understood as being modified in all cases by the term “about.”

“Effective treatment/efficacy”: The efficacy of the MALT1 tumor inhibitor that inhibits the activity of the CARMA3-BCL10-MALT1 complex of the present application in, for example, treating the disorders (e.g., solid tumors) described herein or inducing the responses (e.g., tumor size reduction) described herein can be determined by a skilled clinician. However, treatment is considered “effective treatment” as the term is used herein if, after treatment according to the methods described herein, one or more signs or symptoms of the disorder described herein are altered in a beneficial manner, other clinically acceptable symptoms are improved or even alleviated, or a desired response of at least 10% is induced. Efficacy can be evaluated, for example, by measuring markers, indicators, symptoms, and/or the incidence of the disorder treated according to the methods described herein or any other suitable measurable parameter. Treatment according to the methods described herein can reduce the symptoms or marker levels of the disorder by, for example, at least 10%, at least 15%, at least 20%, at least 25%, at least 30%, at least 40%, at least 50%, at least 60%, at least 70%, at least 80%, or at least 90% or more. Efficacy as described herein can also be measured by failure of the individual to deteriorate as assessed by hospitalization or no longer requiring medical intervention (i.e., disease progression stops). Methods for measuring these indicators are known to those skilled in the art and/or described herein.

When used in conjunction with percentages, “about” can mean±1%.

“Comprising/including/containing” means that other elements may be present in addition to the defined elements. The use of “comprising/including/containing” indicates inclusion rather than limitation.

“Consisting of” refers to the compositions, methods, and their respective components as described herein, excluding any elements not listed in the description of the embodiment.

“Consisting essentially of” refers to the essential elements for a given embodiment. The term allows for the presence of additional elements that do not materially affect the basic and novel or functional characteristics of the embodiment of the present technology.

“Alleviating tumor symptoms” is improving any condition or symptom associated with a tumor. Such a reduction, as measured by any standard technique known to those skilled in the art, is at least 5%, 10%, 20%, 40%, 50%, 60%, 80%, 90%, 95%, 99% or more compared to an equivalent untreated control. Various ways of administering the agents described herein to a subject are known to those skilled in the art.

Unless the context clearly indicates otherwise, “a/the/said” includes plural referents. Similarly, unless the context clearly indicates otherwise, the word “or” is intended to include “and”.

Although methods and materials similar or equivalent to those described herein can be used in the practice or testing of the present application, suitable methods and materials are described below. The abbreviation “e.g.” is derived from the Latin exempli gratia and is used herein to denote non-limiting examples.

Other terms are defined in the description of various aspects and embodiments of the following technologies.

The present invention provides a MALT1 tumor inhibitor, which can inhibit the expression of the MALT1 gene or inactivate the function of the MALT1 protein; the MALT1 tumor inhibitor is selected from agents that can affect the expression of the death domain of the MALT1 protein, agents that knockout the death domain, agents that alter the structure of the death domain, or agents that degrade the death domain.

The antisense oligonucleotides of the present invention are useful for treating or in the preparation of drugs for treating diseases mediated by the MALT1 protein. Human mucosa-associated lymphoid tissue protein 1 (MALT1), also known as human paracaspase-I (HsPCA-I), is a protease discovered from the genomic translocation t(11;18)(q21;q21), a recurrent rearrangement in MALT lymphoma that results in the production of the oncogenic fusion protein API2-MALT1. The MALT1 protein is a multi-domain protein including an N-terminal death domain (DD) and two immunoglobulin (Ig1/2) domains, followed by a paracaspase (PCASP) and a C-terminal Ig3 domain. The constitutive activity of MALT1 protease has been found to drive the survival and proliferation of various lymphoid malignancies. As described earlier, the frequent translocation t(11;18)(q21;q21) leads to the expression of the API2-MALT1 fusion protein in MALT lymphoma, rendering MALT1 protease constitutively active, thereby enhancing anti-apoptotic NF-κB activation in lymphoma tumor cells. In addition, it is a hallmark of many malignant B-cell lymphomas, including activated B-cell (ABC) type diffuse large B-cell lymphoma (DLBCL). The survival and proliferation of ABC-type DLBCL cells are strictly dependent on chronic BCR signaling, which is often triggered by oncogenic driver mutations in the BCR adaptor proteins CD79A and B or the CBM subunit CARD11. In fact, the irreversible tetrapeptide MALT1 inhibitor zVRPR-FMK induces toxicity in ABC DLBCL cells, while other lymphomas, including germinal center B-cell (GCB) type DLBCL cells that are independent of BCR signaling, are insensitive to this MALT1 suicide inhibitor. Furthermore, inhibition of MALT1 protease has been identified as a target for BCR-addicted mantle cell lymphoma (MCL) and chronic lymphocytic leukemia (CLL). The Bruton's tyrosine kinase (BTK) inhibitor ibrutinib has been approved for the treatment of these lymphomas, but primary and secondary drug-induced resistance highlights the need for alternative or combination treatment regimens. MALT1 protease functions downstream of BTK and all known oncogenic events.

Outside the lymphatic system, MALT1 is involved in regulating innate immune responses, growth factor activation, or pro-inflammatory pathways in different types of cells by coupling with different CARMA/CARD scaffold proteins. Therefore, MALT1 is thought to promote the growth and survival of certain tumors that rely on these pathways, including glioblastoma, breast cancer, or melanoma.

Since MALT-1 is a key mediator of the NFκB signaling pathway, it may be a useful drug target for various diseases or disorders. The role of MALT1 protease in generating adaptive immune responses has prompted studies to investigate whether MALT1 inactivation can improve excessive immune responses in autoimmune and inflammatory diseases. Genetic inactivation of MALT1 protease function prevents the development of experimental autoimmune encephalomyelitis (EAE), a mouse model of multiple sclerosis (MS). In addition, MALT1 protease is considered a potential drug target for other autoimmune and inflammatory diseases, including rheumatoid arthritis, psoriasis, Sjögren's syndrome, systemic lupus erythematosus, vasculitic disorders, arthritis, and colitis.

Although MALT1 protease plays a role in activating conventional T cells, its activity is crucial for the development and function of immunosuppressive Treg cells. The development of multi-organ inflammation in mice with defects in MALT1 paracaspase activation suggests that the loss of Treg cells skews the immune system toward destructive autoinflammation, a potential downside of MALT1 protease inactivation. On the other hand, impairment of Treg cell function by MALT1 inhibition is thought to have the potential to enhance anti-tumor immunity in solid cancers.

The present application for the first time discloses another target region of the MALT1 protein different from Para-caspase—a novel target region, the MALT1 tumor effector region.

The present application for the first time demonstrates that the MALT1 tumor effector region located in the death domain of MALT1 is another tumor target region distinct from Para-caspase. Inhibiting the activity of the MALT1 tumor effector region can treat solid tumors.

The MALT1 tumor effector region is not adjacent to the Para-caspase fragment of MALT1. Therefore, the MALT1 tumor inhibitor has a non-competitive advantage over inhibitors targeting the Para-caspase fragment. Thus, the MALT1 tumor inhibitor of the present application can be used in combination with existing MALT1 inhibitors targeting the MALT1 Para-caspase active site to achieve better therapeutic effects.

In certain embodiments of the present invention, the MALT1 tumor inhibitor targets the protein-protein interaction site between MALT1 and BCL-10, thereby reducing or abolishing the binding ability between MALT1 and BCL-10.

In certain embodiments of the present invention, the MALT1 tumor inhibitor can also inhibit MALT1 enzymatic activity.

In certain embodiments of the present invention, the MALT1 tumor inhibitor acts on at least amino acid residues 64 to 114 based on the MALT1 amino acid sequence shown in SEQ ID No. 1, or the nucleotide sequence encoding the same.

The MALT1 protein sequence may have ≥75% homology with the amino acid sequence shown in SEQ ID No. 1, for example, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% homology.

In certain embodiments of the present invention, the MALT1 tumor inhibitor acts on at least amino acid residue 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, and/or 114 of the death domain based on the MALT1 amino acid sequence, or the nucleotide sequence encoding the same.

In certain embodiments of the present invention, the MALT1 tumor inhibitor acts on at least amino acid residue 81 and/or 82 based on the MALT1 amino acid sequence shown in SEQ ID No. 1, or the corresponding codon on the nucleotide sequence encoding the same.

In certain embodiments of the present invention, the MALT1 tumor inhibitor includes an agent that inhibits, blocks, or disrupts the protein-protein interaction between MALT1 and BCL10.

In certain embodiments of the present invention, the agent that inhibits, blocks, or disrupts the protein-protein interaction between MALT1 and BCL10 is selected from covalent cross-linking agents that bind to the MALT1 death domain, polyclonal antibodies against the MALT1 death domain, monoclonal antibodies against the MALT1 death domain, small molecule inhibitors that inhibit the protein-protein interaction between MALT1 and BCL10, reagents for point mutation of the binding site between the MALT1 death domain and BCL10, or reagents for eliminating the binding site between the MALT1 death domain and BCL10 through homologous recombination or gene editing.

In certain embodiments of the present invention, the covalent cross-linking agents that bind to the MALT1 death domain are selected from antioxidants, non-steroidal anti-inflammatory drugs (NSAIDs), natural compounds, proteasome inhibitors, IKK inhibitors, NF-κB-specific antisense oligonucleotides, or cell-penetrating peptides.

In certain embodiments of the present invention, the antioxidants include alantolactone and N-acetylcysteine (NAC).

In certain embodiments of the present invention, the non-steroidal anti-inflammatory drugs (NSAIDs) include cyclooxygenase-2 (COX-2).

In certain embodiments of the present invention, the natural compounds include cantharidin, curcumin, ursolic acid, and resveratrol.

In certain embodiments of the present invention, the proteasome inhibitors include proteasome inhibitor PS341 and melatonin.

In certain embodiments of the present invention, the IKK inhibitor includes the tumor suppressor protein phosphatase PHLPP2.

In certain embodiments of the present invention, the cell-penetrating peptides include the cell-penetrating peptide inhibitor SN50.

In certain embodiments of the present invention, the mutagens for the protein-protein interaction site between MALT1 and BCL10 include: reagents for point mutation of amino acid residue 81 and/or 82 of the MALT1 death domain, CRISPR-Cas9 gene cleavage reagents, and reagents for homologous recombination of the protein-protein interaction site between MALT1 and BCL10.

In certain embodiments of the present invention, the MALT1 tumor inhibitor alters the tumor tissue microenvironment, significantly reducing the number of macrophages in the tumor tissue and converting macrophages in the tumor tissue from M2 type to M1 type.

In certain embodiments of the present invention, the MALT1 tumor inhibitor is an antisense oligonucleotide that inactivates MALT1.

In certain embodiments of the present invention, the nucleotide sequence of the antisense oligonucleotide is as shown in any one of SEQ ID No. 2-6 and 8-12.

The nucleotide sequence of the antisense oligonucleotide may have ≥90% homology with the nucleotide sequence shown in SEQ ID No. 2, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 3, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 4, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 5, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 6, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 8, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 9, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 10, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 11, for example, 90%, 91%, 92%, 93%, 94%, 95%, 96%, 97%, 98%, 99%, or 100% homology.

The nucleotide sequence of the antisense oligonucleotide may have ≥90% homology with the nucleotide sequence shown in SEQ ID No. 12, for example, 90%, 91%, 92%, 93%, 94%, 95%, 96%, 97%, 98%, 99%, or 100% homology.

In certain embodiments of the present invention, the nucleotide sequence of the antisense oligonucleotide is as shown in any one of SEQ ID No. 2-3.

In certain embodiments of the present invention, the agent for degrading the MALT1 death domain is selected from reagents of PROTAC, molecular glue, LYTAC, MODE, ATAC, Apt-LYTAC, AbTAC, PROTAB, REULR, KineTAC, IFLD, ATTEC, AUTAC, or AUTOTAC pathways.

The present invention provides a pharmaceutical composition including an effective amount of the MALT1 tumor inhibitor and a pharmaceutically acceptable carrier.

In certain embodiments of the present invention, the MALT1 tumor inhibitor is an antisense oligonucleotide, whose nucleotide sequence is as shown in any one of SEQ ID No. 2-6 and 8-12.

In certain embodiments of the present invention, the nucleotide sequence of the antisense oligonucleotide is as shown in any one of SEQ ID No. 2-3.

In certain embodiments of the present invention, the antisense oligonucleotide is chemically modified.

In certain embodiments of the present invention, the antisense oligonucleotide further includes modified internucleoside linkages.

In certain embodiments of the present invention, the antisense oligonucleotide further includes modified sugars.

In certain embodiments of the present invention, the antisense oligonucleotide further includes modified nucleobases.

In certain embodiments of the present invention, the antisense oligonucleotide is delivered by a drug delivery system.

In certain embodiments of the present invention, the pharmaceutical composition alters the tumor cell microenvironment, converting macrophages from M2 type to M1 type. The pharmaceutical composition alters the tumor tissue microenvironment, significantly reducing the number of macrophages in the tumor tissue and converting macrophages in the tumor tissue from M2 type to M1 type.

In certain embodiments of the present invention, the pharmaceutical composition further includes a second drug.

In certain embodiments of the present invention, the second drug is selected from immune checkpoint inhibitors, chemotherapeutic agents, or MALT1 enzymatic activity inhibitors.

In certain embodiments of the present invention, the MALT1 enzymatic activity inhibitor is a small molecule inhibitor.

In certain embodiments of the present invention, the immune checkpoint inhibitor is selected from PD-L1, PD-L2, PD-1, CTLA-4, TIM-3, LAG-3, VISTA, or TIGIT inhibitors.

In certain embodiments of the present invention, the PD-1 inhibitor is selected from nivolumab, pembrolizumab, avelumab, cemiplimab, BGB-A317, sintilimab, toripalimab, camrelizumab, or tislelizumab.

In certain embodiments of the present invention, the PD-L1 inhibitor is selected from atezolizumab or durvalumab.

In certain embodiments of the present invention, the CTLA-4 inhibitor is selected from ipilimumab or tremelimumab.

In certain embodiments of the present invention, the chemotherapeutic agent is selected from alkylating agents, antimetabolites, antitumor antibiotics, plant-derived agents, hormonal agents, and other types of chemotherapeutic agents.

In certain embodiments of the present invention, the alkylating agents are selected from nimustine, carmustine, lomustine, cyclophosphamide, ifosfamide, or glyfosfin.

In certain embodiments of the present invention, the antimetabolites are selected from doxifluridine, doxifluridine, 5-fluorouracil, mercaptopurine, thioguanine, fludarabine, tegafur, gemcitabine, ftorafur, hydroxyurea, methotrexate, uftoral, or ancitabine.

In certain embodiments of the present invention, the antitumor antibiotics are selected from dactinomycin D, doxorubicin, daunorubicin, epirubicin, mitomycin, bleomycin, or pirarubicin.

In certain embodiments of the present invention, the antitumor plant and animal-derived agents are selected from irinotecan, harringtonine, hydroxycamptothecin, vinorelbine, paclitaxel, docetaxel, topotecan, vincristine, vindesine, vinblastine, teniposide, etoposide, or elemene.

In certain embodiments of the present invention, the antitumor hormonal agents are selected from exemestane, anastrozole, aminoglutethimide, letrozole, formestane, medroxyprogesterone acetate, tamoxifen, or toremifene.

In certain embodiments of the present invention, the other types of chemotherapeutic agents are selected from asparaginase, carboplatin, cisplatin, dacarbazine, oxaliplatin, eloxatin, lobaplatin, mitoxantrone, or procarbazine.

In certain embodiments of the present invention, the small molecule inhibitor.

In certain embodiments of the present invention, the small molecule inhibitor is selected from compounds having any of the following structural formulas 1-47:

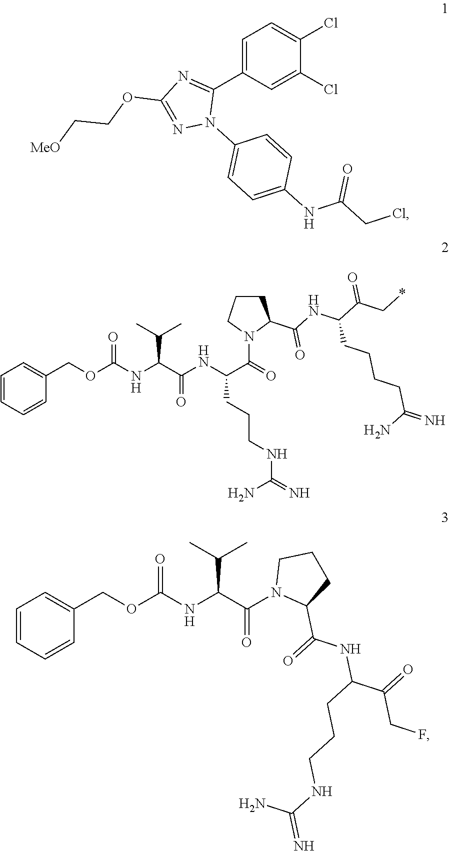

wherein, R is H;

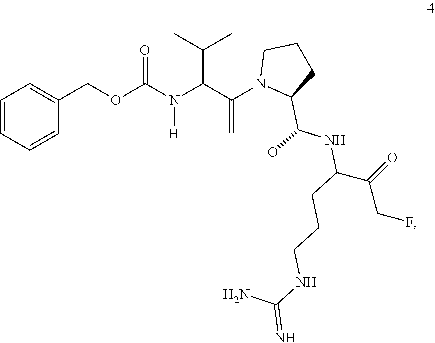

wherein R is methyl;

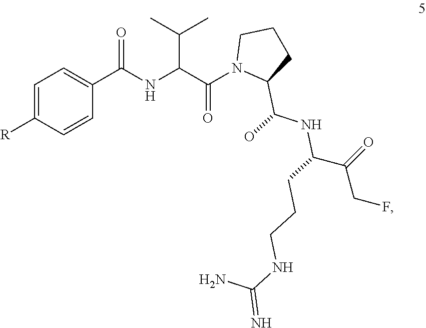

wherein, R is H;

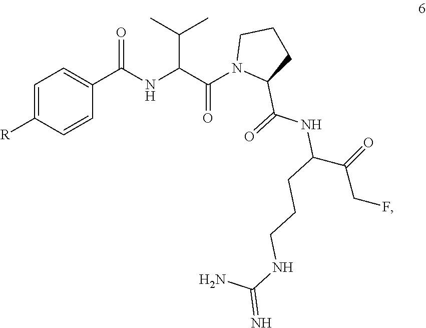

wherein R is Br;

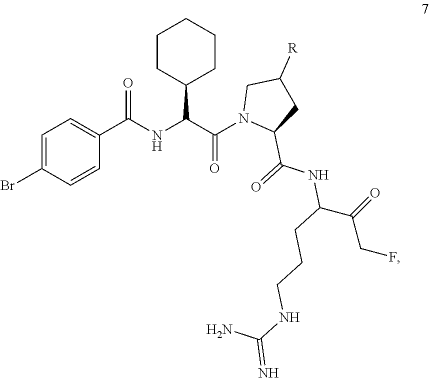

wherein, R is H;

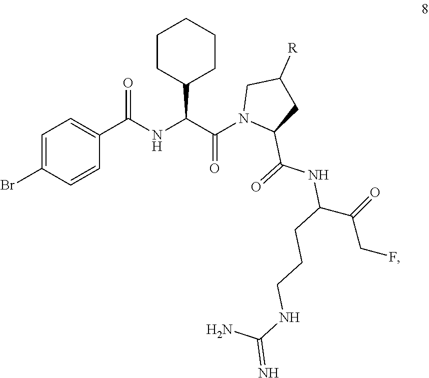

wherein R is OH;

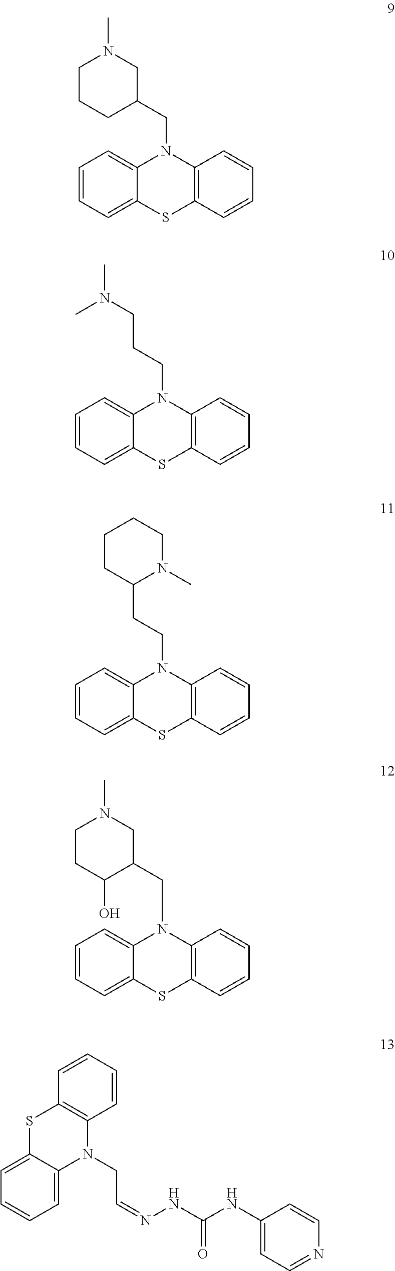

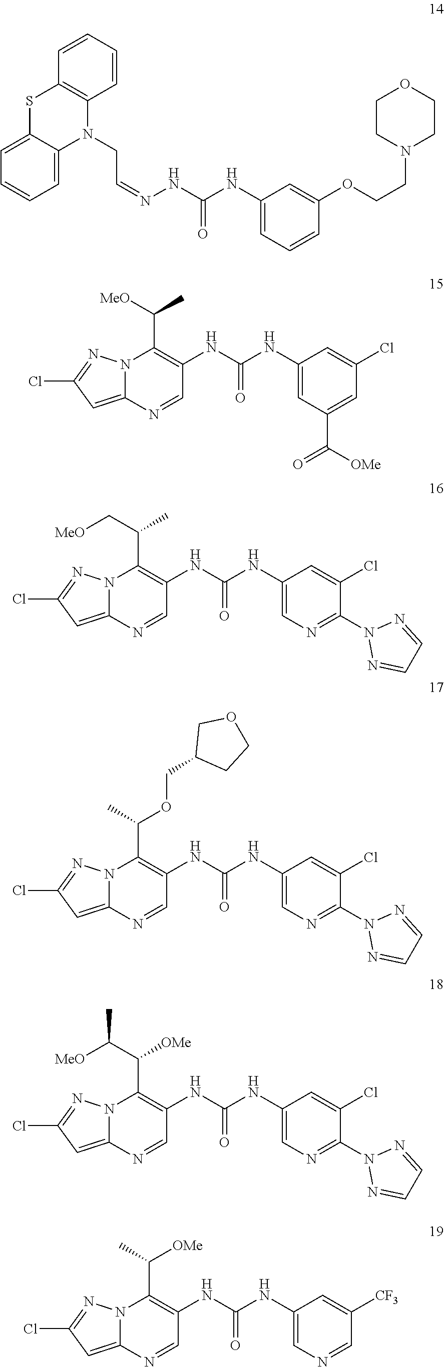

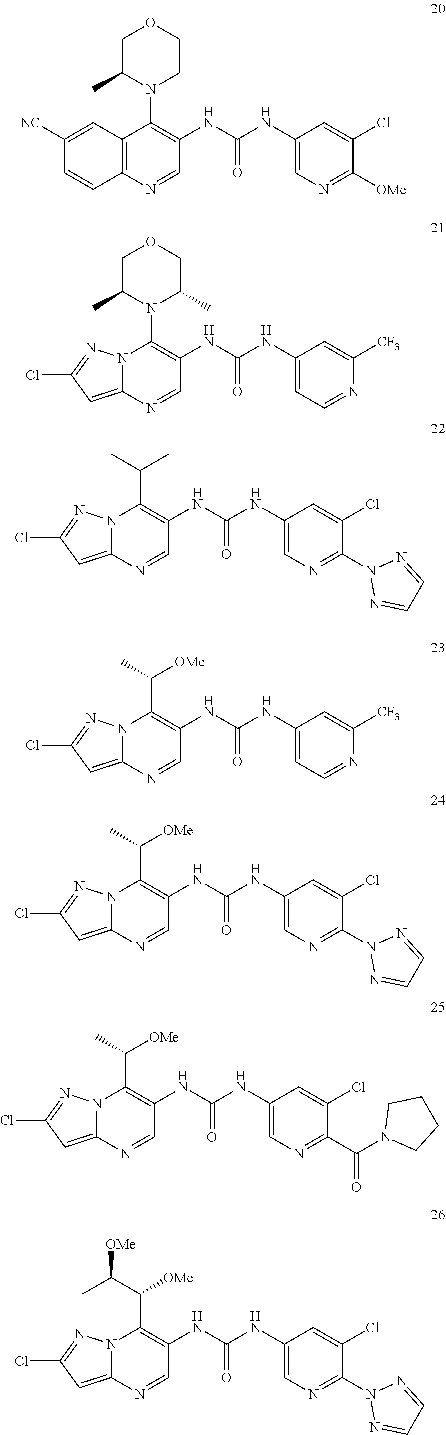

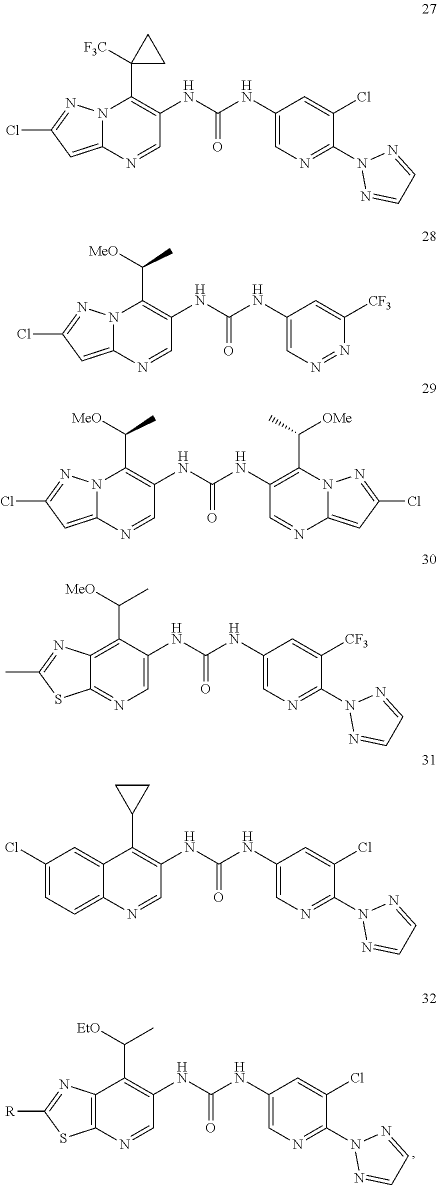

wherein R is methyl;

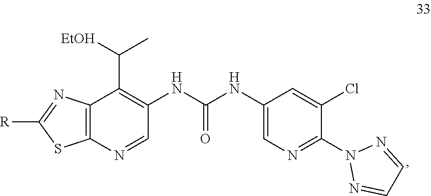

wherein R is H;

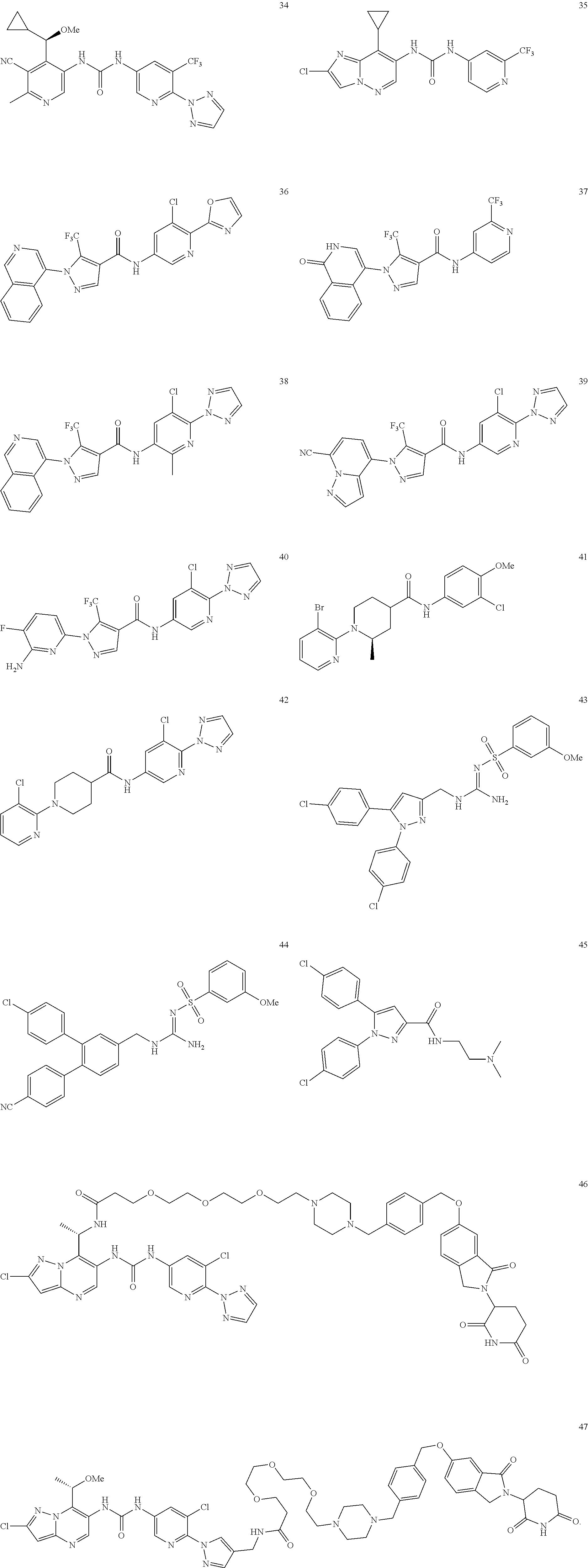

The present invention also provides a drug combination product including an effective amount of the MALT1 tumor inhibitor according to any one of claims 1-13 and a second drug, for simultaneous or sequential administration.

In certain embodiments of the present invention, the MALT1 tumor inhibitor is an antisense oligonucleotide, whose nucleotide sequence is as shown in any one of SEQ ID No. 2-6 and 8-12.

In certain embodiments of the present invention, the nucleotide sequence of the antisense oligonucleotide is as shown in any one of SEQ ID No. 2-3.

In certain embodiments of the present invention, the antisense oligonucleotide is chemically modified.

In certain embodiments of the present invention, the antisense oligonucleotide further includes modified internucleoside linkages.

In certain embodiments of the present invention, the antisense oligonucleotide further includes modified sugars.

In certain embodiments of the present invention, the antisense oligonucleotide further includes modified nucleobases.

In certain embodiments of the present invention, the antisense oligonucleotide is delivered by a drug delivery system.

In certain embodiments of the present invention, the drug combination product alters the tumor tissue microenvironment, significantly reducing the number of macrophages in the tumor tissue and converting macrophages in the tumor tissue from M2 type to M1 type.

In certain embodiments of the present invention, the second drug is selected from immune checkpoint inhibitors or chemotherapeutic agents.

In certain embodiments of the present invention, the immune checkpoint inhibitor is selected from PD-L1, PD-L2, PD-1, CTLA-4, TIM-3, LAG-3, VISTA, or TIGIT inhibitors.

In certain embodiments of the present invention, the PD-1 inhibitor is selected from nivolumab, pembrolizumab, avelumab, cemiplimab, BGB-A317, sintilimab, toripalimab, camrelizumab, or tislelizumab.

In certain embodiments of the present invention, the PD-L1 inhibitor is selected from atezolizumab or durvalumab.

In certain embodiments of the present invention, the CTLA-4 inhibitor is selected from ipilimumab or tremelimumab.

In certain embodiments of the present invention, the chemotherapeutic agent is selected from alkylating agents, antimetabolites, antitumor antibiotics, plant-derived agents, hormonal agents, and other types of chemotherapeutic agents.

In certain embodiments of the present invention, the alkylating agents are selected from nimustine, carmustine, lomustine, cyclophosphamide, ifosfamide, or glyfosfin.

In certain embodiments of the present invention, the antimetabolites are selected from doxifluridine, doxifluridine, 5-fluorouracil, mercaptopurine, thioguanine, fludarabine, tegafur, gemcitabine, ftorafur, hydroxyurea, methotrexate, uftoral, or ancitabine.

In certain embodiments of the present invention, the antitumor antibiotics are selected from dactinomycin D, doxorubicin, daunorubicin, epirubicin, mitomycin, bleomycin, or pirarubicin.

In certain embodiments of the present invention, the antitumor plant and animal-derived agents are selected from irinotecan, harringtonine, hydroxycamptothecin, vinorelbine, paclitaxel, docetaxel, topotecan, vincristine, vindesine, vinblastine, teniposide, etoposide, or elemene.

In certain embodiments of the present invention, the antitumor hormonal agents are selected from exemestane, anastrozole, aminoglutethimide, letrozole, formestane, medroxyprogesterone acetate, tamoxifen, or toremifene.

In certain embodiments of the present invention, the other types of chemotherapeutic agents are selected from asparaginase, carboplatin, cisplatin, dacarbazine, oxaliplatin, eloxatin, lobaplatin, mitoxantrone, or procarbazine.

The present invention also provides applications of the MALT1 tumor inhibitor, the pharmaceutical composition, or the drug combination product in the treatment of diseases, wherein the diseases include tumors.

In certain embodiments of the present invention, the applicable disease is solid tumors; the solid tumors include but are not limited to malignant tumors occurring in the lungs, pancreas, liver, digestive tract, reproductive system and other parts, such as Hodgkin's lymphoma, non-Hodgkin's lymphoma, lung cancer, breast cancer (including triple-negative breast cancer), melanoma, ovarian cancer, gastric cancer, colon cancer, rectal cancer, liver cancer, pancreatic cancer, head and neck malignant tumors, urinary system malignant tumors, endometrial cancer, cervical cancer, osteosarcoma, chondrosarcoma, Ewing's sarcoma, thyroid cancer, hepatoblastoma, and nephroblastoma.

In certain embodiments of the present invention, the route of administration is local administration or intravenous administration.

In certain embodiments of the present invention, the administration method of the pharmaceutical composition of the present invention is selected from intravenous injection, intramuscular injection, subcutaneous injection, rectal perfusion (rectal administration), eye drops, nasal spray, or oral spray (inhalant), and can also be administered topically (surface) or systemically (transdermally) on the skin. The preferred administration method is intravenous injection or subcutaneous injection.

In certain embodiments of the present invention, the indications include solid tumors; the solid tumors include but are not limited to malignant tumors occurring in the lungs, pancreas, liver, digestive tract, reproductive system and other parts, such as Hodgkin's lymphoma, non-Hodgkin's lymphoma, lung cancer, breast cancer (including triple-negative breast cancer), melanoma, ovarian cancer, gastric cancer, colon cancer, rectal cancer, liver cancer, pancreatic cancer, head and neck malignant tumors, urinary system malignant tumors, endometrial cancer, cervical cancer, osteosarcoma, chondrosarcoma, Ewing's sarcoma, thyroid cancer, hepatoblastoma, and nephroblastoma.

In certain embodiments of the present invention, the applicable subject is a mammal.

In certain embodiments of the present invention, the applicable subject is a human.

The present invention also includes a commercial kit including the pharmaceutical composition or drug combination, and instructions for use of the pharmaceutical composition or drug combination, wherein the instructions record the administration method and dosage of the pharmaceutical composition.

The present invention provides a method of treating a disease, which targets the MALT1 death domain, the gene expressing it, or the protein-protein interaction site between MALT1 and BCL-10; the method includes inhibiting, knocking out, altering the structure, or degrading the MALT1 death domain, thereby reducing or abolishing the ability of MALT1 and BCL-10 to form a complex.

In certain embodiments of the present invention, the MALT1 tumor inhibitor, the pharmaceutical composition, or the drug combination product is used as the therapeutic drug.

In certain embodiments of the present invention, the treatment method alters the tumor tissue microenvironment, significantly reducing the number of macrophages in the tumor tissue and converting macrophages from M2 type to M1 type.

In certain embodiments of the present invention, the treatment method is further used in combination with tumor treatment methods selected from surgical treatment, radiation therapy, chemotherapy, or immunotherapy.

In certain embodiments of the present invention, the immunotherapy is selected from non-specific immune stimulation, oncolytic virus therapy, adoptive cell and genetically modified immune cells, tumor vaccines, immune checkpoint monoclonal antibodies (PD1/CTLA4), and T cell-based immunotherapy.

In certain embodiments of the present invention, non-specific immune stimulation includes interferons and interleukins. Interferons enhance the immune system to target cancer cells for programmed cell death and/or slow the growth of cancer cells. Interleukins (e.g., interleukin 2, IL-2, or aldesleukin (Proleukin)) enhance the immune system to produce cells that target cancer cells for programmed cell death. Interleukins are used to treat, for example, kidney cancer and skin cancer, including melanoma. Non-specific immunotherapy can be administered as a single therapy, or can be administered after or simultaneously with another anti-cancer therapy (e.g., chemotherapy or radiation therapy).

In certain embodiments of the present invention, the oncolytic virus therapy uses genetically modified viruses (such as herpes simplex virus or other viruses) to target cancer cells for programmed cell death through immune response.

In certain embodiments of the present invention, the T cell-based immunotherapy is selected from CAR-T therapy, TCR-T therapy, TILs therapy, or CTL therapy.

In certain embodiments of the present invention, the pharmaceutical composition described in the present application is administered to the patient in combination with CAR-T cells targeting the following substances: EGFR (epidermal growth factor receptor) on non-small cell lung cancer, epithelial cancer, and glioma; EGFRvIII (variant III of epidermal growth factor receptor) on glioblastoma; HER2 (human epidermal growth factor receptor 2) on ovarian cancer, breast cancer, glioblastoma, colon cancer, osteosarcoma, and medulloblastoma; MSLN (mesothelin) on mesothelioma, ovarian cancer, and pancreatic adenocarcinoma; PSMA (prostate-specific membrane antigen) on prostate cancer; CEA (carcinoembryonic antigen) on pancreatic adenocarcinoma, breast cancer, and colorectal epithelial cancer; GD2 (disialoganglioside 2) on neuroblastoma and melanoma; IL13Rα2 (interleukin-13Ra2) on glioma; GPC3 (Glypican-3) on hepatocellular carcinoma; CAIX (carbonic anhydrase IX) on renal cell carcinoma (RCC); L1-CAM (L1 cell adhesion molecule) on neuroblastoma, melanoma, and ovarian adenocarcinoma; CA125 (cancer antigen 125, also known as MUC16) on epithelial ovarian cancer; CD133 (cluster of differentiation 133, also known as prominin-1) on glioblastoma and cholangiocarcinoma (CCA); FAP (fibroblast activation protein) on malignant pleural mesothelioma (MPM); CTAG1B (cancer/testis antigen 1B, also known as NY-ESO-1) on melanoma and ovarian cancer; MUC1 (mucin 1) on seminal vesicle cancer; FR-α (folate receptor-α) on ovarian cancer.

In certain embodiments of the present invention, the disease includes tumors, further solid tumors.

In certain embodiments of the present invention, the solid tumors include but are not limited to malignant tumors occurring in the lungs, pancreas, liver, digestive tract, reproductive system and other parts, such as Hodgkin's lymphoma, non-Hodgkin's lymphoma, lung cancer, breast cancer (including triple-negative breast cancer), melanoma, ovarian cancer, gastric cancer, colon cancer, rectal cancer, liver cancer, pancreatic cancer, head and neck malignant tumors, urinary system malignant tumors, endometrial cancer, cervical cancer, osteosarcoma, chondrosarcoma, Ewing's sarcoma, thyroid cancer, hepatoblastoma, and nephroblastoma.

In certain embodiments of the present invention, the subject of the treatment method is a mammal.

In certain embodiments of the present invention, the subject of the treatment method is a human.

The present invention also provides a target, which is the protein-protein interaction site between MALT1 and BCL-10, or the nucleotide sequence encoding the same.

In some embodiments of the present invention, the target is amino acid residues 64 to 114 based on the MALT1 amino acid sequence shown in SEQ ID No. 1, or the nucleotide sequence encoding the same.

In some embodiments of the present invention, the target is amino acid residue 81 and/or 82 based on the MALT1 amino acid sequence shown in SEQ ID No. 1, or the corresponding codon on the nucleotide sequence encoding the same.

The present invention also provides an application of the target in drug screening, wherein drugs for treating tumors act on the target, and the drugs include inhibitors of the protein-protein binding between MALT1 and BCL-10.

The present invention also provides an inhibitor that acts on the target and inhibits the interaction between MALT-1 and BCL10.

To make the technical means, creative features, objectives, and effects of the present invention easy to understand, the technical solutions in the embodiments of the present invention will be clearly and completely described below. Obviously, the described embodiments are only a part of the embodiments of the present invention, not all of them. Based on the embodiments of the present invention, all other embodiments obtained by those of ordinary skill in the art without creative work shall fall within the protection scope of the present invention.

In the embodiments of the present invention, the mouse MALT1-1 protein and its mRNA, and the human MALT1-1 protein and its mRNA are taken as examples to illustrate the function of the tumor effector region on the MALT1-1 death domain and the role of the MALT1 tumor inhibitor.

### Example 1

In this example, the effect of different mutation sites of MALT1 death domain gene mutants on tumor growth was determined using an orthotopic injection model of mammary fat pad. The amino acid sequence of the mouse MALT1 protein is shown in SEQ ID No. 22 (NCBI Reference Sequence: NP_766421.1).

The experimental results are shown in FIG. 5. It can be seen from FIG. 5 that overexpression of the MALT1 Para-caspase active center gene mutation promoted the growth of tumor cells. However, on the premise that the MALT1 Para-caspase active center is mutated, such as the mutation of valine at position 87 and/or leucine at position 88 of the MALT1 death domain (corresponding to lanes D and E in FIG. 5), the overexpression of the MALT1 Para-caspase active center gene mutation lost the effect of promoting tumor growth.

This experiment indicates that the amino acid residues at positions 87 and/or 88 of the MALT1 death domain play a key role in promoting tumor growth by other mutations of MALT1, which correspond to amino acid residues 81 and/or 82 of the human MALT-1 death domain amino acid sequence (e.g., the human MALT-1 death domain amino acid sequence shown in SEQ ID No. 1 (NCBI Reference Sequence: NP_006776.1)).

### Example 2

In this experimental example, co-immunoprecipitation (IP) was used to study and analyze the interaction site between MALT1 and BCL10.

Co-immunoprecipitation is a method used to study protein-protein interactions. This method can selectively enrich specific protein complexes and investigate the protein components therein and the relationships between them.

Specifically, for MALT1 and BCL10, it is first necessary to prepare monoclonal antibodies to specifically recognize the two proteins respectively, and then combine these two antibodies with the corresponding antigen proteins (i.e., MALT1 and BCL10) to form MALT1-antibody and BCL10-antibody complexes. Next, these complexes are incubated with cell lysate to allow non-specific binding of the above complexes to other components. The proteins not specifically bound to the above complexes can be washed away through subsequent centrifugation and washing processes. Finally, the obtained co-immunoprecipitated complexes can be used for Western blot detection to determine whether MALT1 and BCL10 bind through interaction. If MALT1 and BCL10 bind, the complex of the two should be effectively enriched, and the results of Western blot testing will show a high degree of correlation. On the contrary, if the complex cannot be sufficiently enriched or the detection result is negative, it can indicate that there is no interaction between the two.

### The Specific Operations are as Follows:

### 2.1 Cell Preparation

S1. Infect E0771-TR with lentivirus of pLKO.1-puro MALT1 shRNA1, and obtain E0771-TR MALT1-KD cells after selection with Puromycin. S2. Infect E0771-TR with retrovirus MSCV-Puro-MALT1-wild type, and obtain E0771-TR MALT1-wild type overexpressing cells after selection with Puromycin. S3. Infect E0771-TR with retrovirus MSCV-Puro-MALT1-C83A, and obtain E0771-TR MALT1-C83A overexpressing cells after selection with Puromycin. S4. Infect E0771-TR with retrovirus MSCV-Puro-MALT1-V87R, and obtain E0771-TR MALT1-V87R overexpressing cells after selection with Puromycin. S5. Infect E0771-TR with retrovirus MSCV-Puro-MALT1-L88D, and obtain E0771-TR MALT1-L88D overexpressing cells after selection with Puromycin. S6. Infect E0771-TR with retrovirus MSCV-Puro-MALT1-E104R, and obtain E0771-TR MALT1-E104R overexpressing cells after selection with Puromycin. S7. Infect E0771-TR with retrovirus MSCV-Puro-MALT1-C461A, and obtain E0771-TR MALT1-C461A overexpressing cells after selection with Puromycin. S8. Infect E0771-TR with retrovirus MSCV-Puro-MALT1-C83A-C461A, and obtain E0771-TR MALT1-C83A-C461A overexpressing cells after selection with Puromycin. S9. Infect E0771-TR with retrovirus MSCV-Puro-MALT1-V87R-C461A, and obtain E0771-TR MALT1-V87R-C461A overexpressing cells after selection with Puromycin. S10. Infect E0771-TR with retrovirus MSCV-Puro-MALT1-L88D-C461A, and obtain E0771-TR MALT1-L88D-C461A overexpressing cells after selection with Puromycin. S11. Infect E0771-TR with retrovirus MSCV-Puro-MALT1-E104R-C461A, and obtain E0771-TR MALT1-E104R-C461A overexpressing cells after selection with Puromycin.

2.2 Infect the 11 Cell Lines Obtained in 2.1 with Lentivirus pLVX-BCL10-3λFlag-Blast Respectively, and Obtain the Following 11 Cell Lines after Selection with Blasticidin:

1.BCL10 with three repeated FLAGs overexpressed in E0771-TR MALT1-KD cells (negative control); 2. BCL10 with three repeated FLAGs overexpressed in E0771-TR MALT1-wild type overexpressing cells (positive control); 3. BCL10 with three repeated FLAGs overexpressed in E0771-TR MALT1-C83A overexpressing cells; 4. BCL10 with three repeated FLAGs overexpressed in E0771-TR MALT1-V87R overexpressing cells; 5. BCL10 with three repeated FLAGs overexpressed in E0771-TR MALT1-L88D overexpressing cells; 6. BCL10 with three repeated FLAGs overexpressed in E0771-TR MALT1-E104R overexpressing cells; 7. BCL10 with three repeated FLAGs overexpressed in E0771-TR MALT1-C461A overexpressing cells; 8. BCL10 with three repeated FLAGs overexpressed in E0771-TR MALT1-C83A-C461A overexpressing cells; 9. BCL10 with three repeated FLAGs overexpressed in E0771-TR MALT1-V87R-C461A overexpressing cells; 10. BCL10 with three repeated FLAGs overexpressed in E0771-TR MALT1-L88D-C461A overexpressing cells; 11. BCL10 with three repeated FLAGs overexpressed in E0771-TR MALT1-E104R-C461A overexpressing cells.

2.3 Plate the 11 Cell Lines Obtained in 2.2 into 6-Well Plates at a Density of 5×10⁵ Cells Per Well, and Lyse the Cells with Cell Lysate after 24 Hours.

The formula of the cell lysate is as follows:

10 mM Tris, pH 7.4; 100 mM NaCl; 1 mM EDTA; 1 mM EGTA; 1 mM NaF; 20 mM Na4P2O7; 2 mM Na3VO4; 1% Triton X-100; 10% glycerol; 0.1% SDS; 0.5% deoxycholate.

### 2.4 the Co-Immunoprecipitation Process is Specifically as Follows:

### 2.4.1 Bead Preparation

1. Prepare 3 μL of concentrated beads for each cell lysate sample from the 6-well plate.

2. Transfer the beads to each 1.5 mL microcentrifuge tube and wash the beads 3 times with cell lysis buffer:

(1) Resuspend the beads with 200 μL of cell lysis buffer.

(2) Centrifuge at 14,000 g for 30 seconds, and remove the supernatant with the help of a magnetic stand.

### 2.4.2 Immunoprecipitation and Incubation

1. Add 160 μL of cell lysate to each tube at a ratio of 200 μL of cell lysate per sample, where 160 μL is used for IP and 40 μL is used as input.

2. (Optional) Add 640 μL of cell lysis buffer (with protease inhibitor cocktail) to each tube to allow more thorough mixing.

3. Invert the tubes overnight at 4° C.

### 2.4.3 Washing and Elution

1. Centrifuge the tubes at 14,000 g for 30 seconds, and remove the supernatant with the help of a magnetic stand.

2. Wash the beads 5 times with 500 μL of cell lysis buffer (with protease inhibitor cocktail) per tube:

(1) Add 500 μL of cell lysis buffer to each tube; (2) Invert and rotate the tubes at 4° C. for 10 minutes; (3) Centrifuge at 14,000 g for 30 seconds, and remove the supernatant with the help of a magnetic stand.

3. Resuspend the beads with 60-100 μL of SDS loading buffer.

4. Heat the mixture at 95° C. for 5 minutes. Centrifuge at 14,000 g for 1 minute.

5. The supernatant is the prepared SDS-PAGE loading sample.

### 2.4.4 Perform SDS-PAGE Electrophoresis on the 11 Samples in 2.4.3

Samples numbered 1-11 are the lysed samples of the 11 cell lines in 2.2 respectively. The results in FIG. 7 indicate that mutations of V87R and L88D in the MALT1 death domain affect the binding between MALT1 and BCL10 (the intensity of the red box in FIG. 7 is basically consistent with that of the blue box in the case of MALT1 knockdown). In addition, cell lines 7-11 show the same trend as cell lines 2-6, proving that the binding between MALT1 and BCL10 is independent of the Para-caspase fragment mutation (C461A) of MALT1.

This experiment indicates that the amino acid residues at positions 87 and/or 88 in the MALT1 death domain play a key role in the binding between MALT1 and BCL10.

### Example 3

This example provides a method for co-culturing tumor cells-macrophages-somatic cells to prove that mutations of V87R and L88D weaken the effect of the death domain on macrophages.

### 3.1 Isolation and Culture of Macrophages

1. Sacrifice 10-12 week-old mice (gender needs to be recorded), separate the bones of the two hands and two feet, and soak them in PBS. 2. Cut off both ends of the bones, use a 10 mL syringe fitted with a 1 ml syringe needle to flush the bone marrow with DMEM regular into a 50 mL tube with a 70 μm sterile cell sieve (about 20 mL of medium is used for 6 small bones per mouse). 3. Centrifuge at 300 g for 3 minutes. 4. Discard the supernatant, add 2 mL of 1× red blood cell lysis buffer and react at room temperature for 1 minute, then immediately add 5 mL of DMEM regular to terminate. 5. Centrifuge at 300 g for 3 minutes. 6. Discard the supernatant, add 2 mL of PBS to wash once. 7. Centrifuge at 300 g for 3 minutes. 8. Discard the supernatant, add 2 mL of BMDM medium (DMEM+20% BI FBS+10 ng/ml M-CSF+HEPES (100×)+GLUTMAX (100×)) to resuspend, and filter through a 70 μm sterile cell sieve again. 9. Count the cells, adjust the cell concentration to 2×10⁶ cells/mL with BMDM medium, and add 10 mL to a 10 cm dish. 10. Incubate at 37° C. for 48 hours. 11. Change the medium for the first time after 48 hours: aspirate 8 mL of medium, leave about 2 mL, then add 10 mL of BMDM medium (DMEM+20% BI FBS+10 ng/mL M-CSF+HEPES (100×)+GLUTMAX (100×)), and continue incubating at 37° C. for 24 hours. 12. Change the medium for the second time after 24 hours: aspirate all the medium, wash once with PBS, then add 10 mL of BMDM medium (DMEM+20% BI FBS+20 ng/ml M-CSF+HEPES (100×)+GLUTMAX (100×)), and continue incubating at 37° C. for 24 hours. 13. After 24 hours, the macrophages have been cultured for 96 hours. Take out the culture dish, wash twice with PBS, add 3 mL of trypsin and digest at room temperature for 3 minutes, then aspirate the trypsin (this step is to remove monocytes). 14. Add 3 mL of trypsin and digest at 37° C. for 12 minutes, do not add medium to terminate, directly blow up the cells with trypsin, and add to a 15 mL tube containing 5 mL of DMEM regular medium. Centrifuge at 300 g for 3 minutes. 15. Discard the supernatant, add 1 mL of PBS to resuspend, centrifuge at 300 g for 3 minutes. 16. Discard the supernatant, add 1 mL of DMEM regular medium to resuspend, count, and adjust to the required concentration.

### 3.2 Isolation and Culture of T Cells

1. Coat one well of a 6-well plate with anti-CD3 and anti-CD28 8-12 hours in advance: add 1 mL of PBS, 1 μL of anti-CD3 and 2 μL of anti-CD28, mix well and place in a 4° C. refrigerator. 2. Sacrifice 6-8 week-old OT-1 mice (same gender as the wild-type mice in the same experiment), separate the spleen and mammary lymph nodes, and soak them in PBS. 3. Transfer the spleen and lymph nodes to a 1.5 mL EP tube containing 200 μL of 1640 regular in a ultra-clean bench. 4. Mince the spleen and lymph nodes, and filter the cell suspension through a 50 mL tube with a 70 μm sterile cell sieve. 5. Centrifuge at 300 g for 3 minutes. 6. Discard the supernatant, add 2 mL of 1× red blood cell lysis buffer and react at room temperature for 1 minute, then immediately add 5 mL of 1640 regular medium to terminate. 7. Centrifuge at 300 g for 3 minutes. 8. Discard the supernatant, add 2 mL of PBS to wash once. 9. Centrifuge at 300 g for 3 minutes. 10. Discard the supernatant, add 2 mL of PBS to resuspend, and filter through a 70 μm sterile cell sieve again. 11. Count the cells, adjust the cell concentration to 1×10⁸ cells/mL with 1× MoJoSort buffer. 12. Take out 200 μL of the cell suspension for CD8 T cell isolation. 13. Wash the isolated cells once with PBS, then add 2 mL of TCM (1640+20% BI FBS+20 ng/ml IL2+HEPES (100×)+GLUTMAX (100×)+sodium pyruvate (100×)+beta-mercaptoethanol (1000×)) to resuspend. 14. Take out the coated plate, aspirate the liquid, wash once with 1 mL of PBS, add the cell suspension, and incubate at 37° C. for 48 hours. 15. After 48 hours, transfer the T cells in the 6-well plate to a 10 cm dish, and incubate at 37° C. for 24 hours. 16. After 24 hours, transfer the T cells in the 10 cm dish to a new 10 cm dish at a ratio of 1:3, and incubate at 37° C. for 24 hours. 17. After 24 hours, the T cells have been cultured for 96 hours and are in the best state for use. 18. Before adding T cells, incubate with 100 ng/mL OVA for 2 hours. 19. Aspirate the medium containing OVA, and add DMEM regular medium containing a specific number of T cells (with or without T cells).

### 3.3 Preparation of Tumor Cell Culture Medium

1. Count the cells with DMEM regular medium and plate them. 2. Change the medium for the first time 48 hours after adding macrophages, using regular medium.

1. Incubate with 100 ng/mL OVA 2 hours before adding T cells, then aspirate the OVA and add new medium containing T cells or not. 4. After 24 hours, aspirate the medium, add 500 μL of 1× lysis buffer to each well of the 6-well plate, and 200 μL of 1× lysis buffer to each well of the 24-well plate.

### 3.4 Determine the Survival Rate of Tumor Cells Using Luciferase Assay

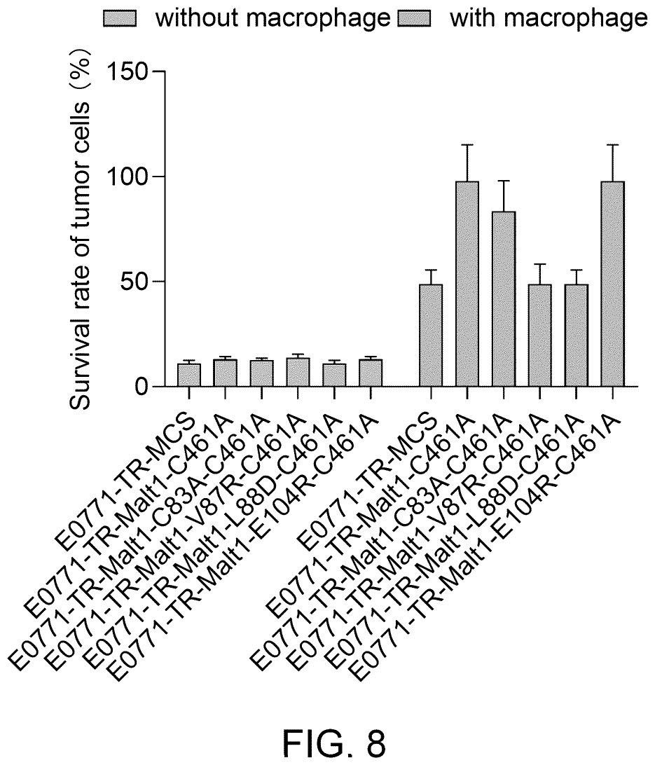

The results are shown in FIG. 8, which shows the tumor cell survival rates in 11 cell lines before and after the addition of macrophages.

The experimental results indicate that there is no difference in tumor cell survival rates among cell lines 1-6, suggesting that the presence of corresponding MALT1 mutants cannot enhance tumor cell survival in the case of only T cells.

Macrophages themselves can increase tumor cell survival rate (comparison between 7 and 1). Once macrophages are added, the comparison of tumor cell survival rates between cell lines 7-12 (with macrophages added) and cell lines 8, 9, and 12 on the right indicates that MALT1 further enhances the protective effect of macrophages on tumors (comparison of tumor cell survival rates between cell lines 8, 9, 12 and cell line 7). However, when the amino acid residues at positions 87 and/or 88 in the MALT1 death domain are mutated, the protective effect of MALT1 disappears, and at this time, the tumor cell survival rates of cell lines 7, 10, and 11 are basically consistent.

This experiment indicates that MALT1 plays a role in protecting tumor cells by enhancing the protective effect of macrophages on tumors; and this protective effect depends on the structure formed by the amino acid residues at positions 87 and/or 88 of the death domain.

### Example 4

This example studies the effect of cell-conditioned medium on macrophage proliferation.

### 4.1 Cell-Conditioned Medium

Plate 2×10⁶ cells into 10 cm dishes with DMEM regular medium (DMEM+10% FBS+1% PS), collect the medium after 36 hours, centrifuge at 3500 g for 10 minutes, and filter the supernatant with a 0.45 μm filter membrane to obtain conditioned medium.

4.2 Method for Culturing Macrophages with Cell-Conditioned Medium

Add 1 mL of 1×10⁶ cells/mL macrophage suspension to each well of a 6-well plate (using macrophage medium: DMEM+10% FBS+1% PS+1% Glut MAX+10 ng/ml M-SCF at this time), then add 1 mL of conditioned medium to each well, mix well, and count after culturing for 48 hours.

### 4.3 Method for Determining Cell Number

Digest macrophages with trypsin at 37° C. for 15 minutes, centrifuge at 300 g for 3 minutes, discard the supernatant, dilute to an appropriate multiple, stain with trypan blue, and count with a hemocytometer.

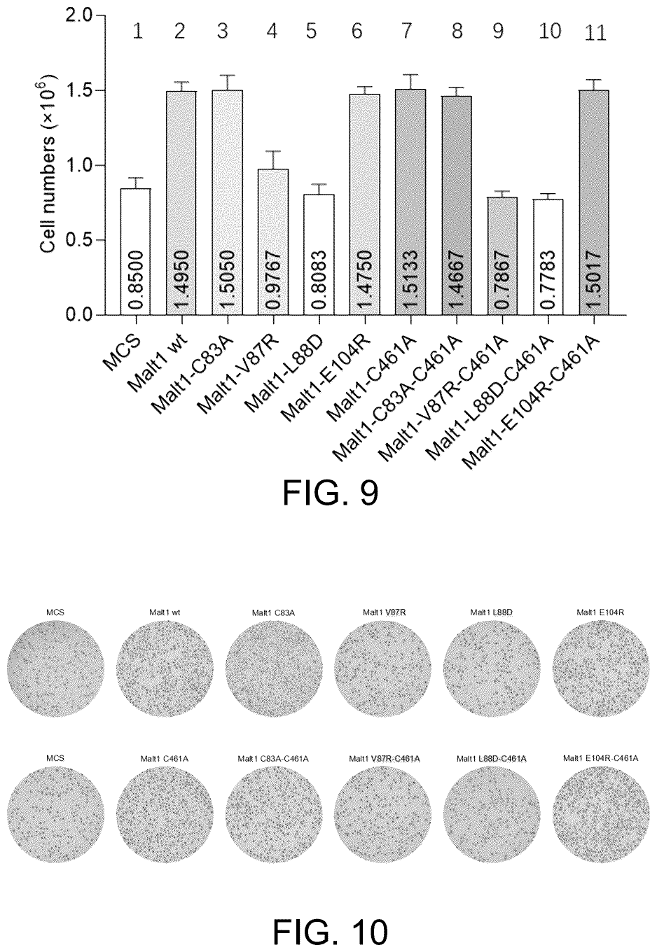

The results are shown in FIGS. 9 and 10, where FIG. 10 is the visualized result of cell counts obtained by direct photography under a microscope.

Cell lines 3-6 are mutated under the MALT1 wild type, and cell lines 8-11 are mutated on the basis of MALT1 Para-caspase active center mutation. Comparison between cell line 2, cell line 7 and cell line 1 indicates that the effect of MALT1 on macrophage proliferation is independent of Para-caspase activity. However, once V87R (cell lines 4 and 9) and L88D (cell lines 5 and 10) are mutated, the effect of MALT1 on macrophage proliferation disappears immediately (comparison between cell lines 4, 5 and cell line 2; comparison between cell lines 9, 10 and cell line 7).

This experiment indicates that the amino acid residues at positions 87 and/or 88 in the MALT1 death domain weaken the effect of the MALT1 death domain on macrophages.

### Example 5

This example studies the effect of mutations in the MALT1 death domain gene on T cell killing ability.

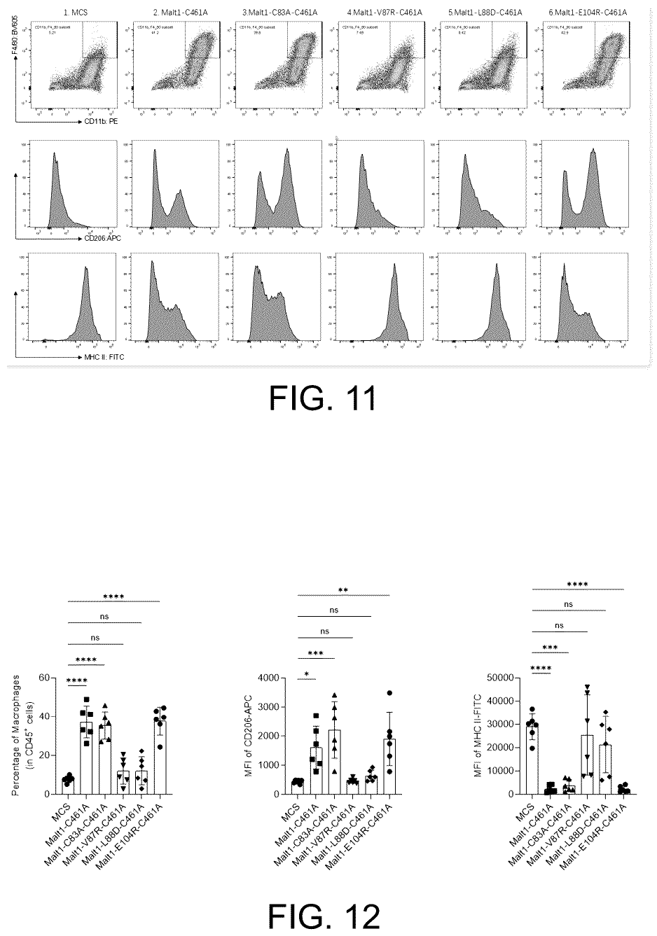

The experimental results are shown in FIG. 11, which is a single-parameter histogram of flow cytometry. Column 1 is the control group, column 2 is the MALT1 wild type group, and columns 3-6 are mutated under the MALT1 wild type group. It can be seen that there is no significant difference between columns 3-6 and column 2, indicating that mutations in the MALT1 death domain gene have no effect on the killing of T cells alone.

The results prove that mutations of the amino acid residues at positions 87 and/or 88 in the MALT1 death domain do not affect the protection of tumor cells by MALT1 Para-caspase in the case of only T cells.

This experiment indicates that the structure formed by the amino acid residues at positions 87 and/or 88 in the MALT1 death domain is independent of the effect of MALT1 Para-caspase on T cells.

### Example 6

In this example, flow cytometry was used to detect the infiltration of tumor immune cells in mice.

The experimental results are shown in FIGS. 11 and 12.

FIG. 11 shows the results of flow cytometry. In the figure, 1 is the control cell line, 2 is the cell line with MALT1 Para-caspase active center mutation, and 3-6 are cell lines with other mutations on the basis of 2. When the above cell lines were injected into the mammary fat pad of mice, the tumors were taken for flow cytometry analysis on day 15. The upper right corner of the first row of pictures indicates macrophages in the tumor. It can be seen that compared with 1, macrophages infiltration increases in 2, 3, and 6, but mutations in 4 (V87R) and 5 (L88D) make the macrophages in the tumor basically return to a level similar to 1.

In FIG. 12, the left graph shows the corresponding relationship between mutant strains at different sites of the MALT1 death domain and macrophage ratio, the middle graph shows the corresponding relationship between mutant strains at different sites of the MALT1 death domain and CD206 intensity (M2 marker), and the right graph shows the corresponding relationship between mutant strains at different sites of the MALT1 death domain and MHCI intensity (M1 marker). Simply put, M2 promotes tumors and M1 inhibits tumors. It can be seen that mutations in 4 (V87R) and 5 (L88D) reduce M2 and increase M1 in tumor-infiltrating macrophages, while 2, 3, and 6 show a high proportion of M2 and a low proportion of M1.

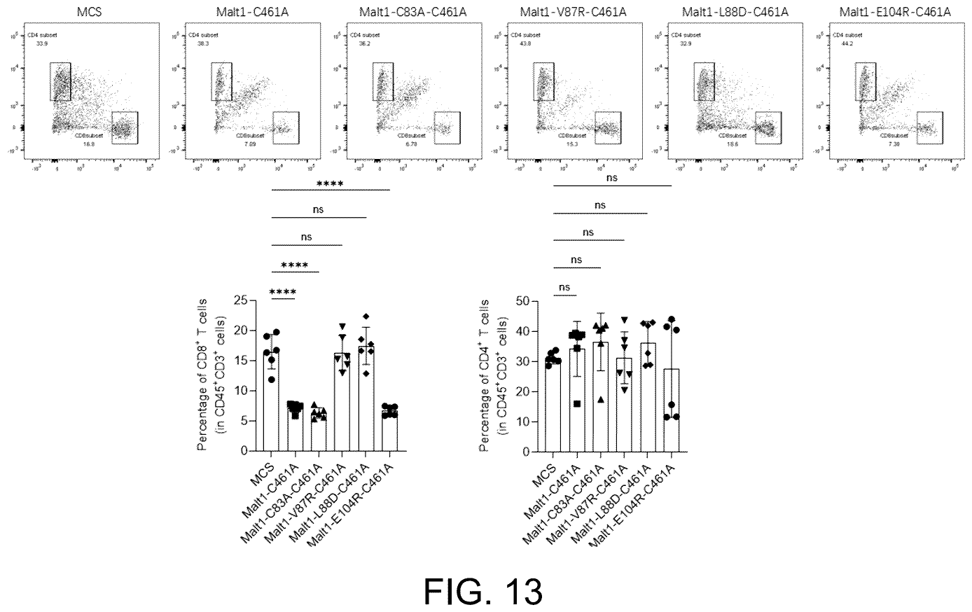

FIG. 13 shows the T cell infiltration of tumors in each mouse detected by flow cytometry. In the lower graph, column 1 is the control group, column 2 represents the MALT1 Para-caspase active center mutation, and columns 3-6 are cell lines with other mutations on the basis of column 2. When these cells were injected into the mammary fat pad of mice, the tumors were taken for flow cytometry analysis on day 15. The upper left corner of the first row of pictures indicates CD4 T cells, and the lower right corner of the first row of pictures indicates CD8 T cells. Compared with column 1, CD8 T infiltration decreases in columns 2, 3, and 6, but mutations in 4 (V87R) and 5 (L88D) make the CD8 T infiltration in the tumor basically return to a level similar to column 1. However, these mutations have little effect on CD4.

The results indicate that mutations of V87R and L88D in the MALT1 death domain weaken the effect of the MALT1 death domain on macrophages, thereby weakening the effect of MALT1 on reducing CD8 T cell infiltration, which is still valid in the case of in vivo tumors.

This experiment indicates that the structure formed by the amino acid residues at positions 87 and/or 88 of the MALT1 death domain can protect tumor cells in animals, and this effect is based on its influence on macrophages.

It can be concluded from the foregoing examples that there is a structure in the MALT1 protein that plays a protective role in the growth of tumor cells, and this protective effect is derived from the influence of this structure on macrophages in the tumor microenvironment. The amino acid residues at positions 87 and/or 88 of the MALT1 protein death domain play a key role in this structure, and these amino acid residues are also the key sites for the binding between MALT1 and BCL10.

It can also be concluded from the foregoing examples that mutating the amino acid residues at positions 87 and/or 88 of the MALT1 protein can destroy this structure, thereby disrupting the protective effect of the MALT1 protein on the growth of tumor cells and achieving the effect of inhibiting tumor growth. Disrupting the binding between MALT1 and BCL10 can also disrupt the protective effect of the MALT1 protein on the growth of tumor cells, thereby achieving the effect of inhibiting tumor growth.

It should be noted that in the present application, the type and form of the MALT1 tumor inhibitor are not specifically limited, including but not limited to those listed above, and can be specifically selected according to actual conditions.

In the present application, the MALT1 tumor inhibitor that inhibits the activity of the MALT1 tumor effector region can be used to treat all cancers, and should not be limited to the types of cancers listed in the present application document.

### Example 7

Preparation of antisense oligonucleotides inhibiting MALT1. The strategy of antisense nucleic acids includes the following steps:

S1. Considering the specificity between the antisense nucleic acid and the target nucleic acid and the hairpin-like structure that may be formed by the base complementarity within the nucleotide sequence of the antisense nucleic acid, the nucleotide sequence of the antisense nucleic acid needs to target the amino acid residues at positions 81 and/or 82 of the death domain (DD) of MALT1; S2. Considering the stability of hybridization and the membrane permeability of the antisense oligonucleotide, select the length of the antisense oligonucleotide, which ranges from 15 to 40 bases; S3. Considering the affinity of the antisense oligonucleotide for the target sequence, since the affinity of GC base pairs is greater than that between AU, increasing the ratio of GC base pairs to AU base pairs in the antisense oligonucleotide can appropriately reduce the length of the antisense oligonucleotide; S4. Set a control, which can be missense or reverse; S5. Use the designed antisense oligonucleotides to inhibit the expression of the same target sequence MALT-1 mRNA, and screen the antisense oligonucleotide with the best inhibitory effect.

The specific steps for screening the antisense oligonucleotide with the best inhibitory effect are as follows:
a. Transfect 2×10⁵ E0771 cells into each well of a 12-well plate and incubate overnight;
b. On the day after operation a, transfect the corresponding ASO drug with 5 μL of Lipofectamine™ 3000 until its final concentration is 50 nM;
c. 6 hours after operation b, replace the serum-free medium with medium containing 10% serum;
d. 24 hours after operation c, replace the serum-free medium again;
e. 48 hours after operation d, harvest the obtained protein and perform Western blot experiment.

The Western blot results of antisense oligonucleotides targeting E0771 cells after transfection with Lipofectamine™ 3000 are shown in FIG. 5: in this figure, 48 hours after transfection with Lipofectamine™ 3000, 5 μL of Lipofectamine™ 3000 was added to each well of the 12-well plate.

TABLE 1
Specific Sequences of Each ASO in
the Western Blot Assay

| Malt1 ASO ID | Malt1 ASO sequence |
|---|---|
| A | AGCCTTGGCCCACTGTAGATT |
| B | TCATGAGCCTTGGCCCACTG |
| C | AAGTTCATGAGCCTTGGCCC |
| D | GAAGTTCATGAGCCTTGGCCC |
| E | GGTTTGTGGACTATACTTGT |
| F | GGTGGTTTGTGGACTATACTTG |
| G | TTGTGGACTATACTTGTGTA |
| H | TAGAGGCAGTGCTTGGGAAG |
| I | GAGGCAGTGCTTGGGAAGGTC |
| J | GTCCAGCCAACACTGCCTTG |
| K | CAAGAGCAGATTTATTTTGT |
| L | TGATCTTACCATATACTCCT |
| M | TTACAATGATCTTACCATATA |
| N | AATATAACAATCCTGGGAGT |
| C | AATATAACAATCCTGGGAGTGGGG |
| P | ATTTTACCTTTTCCGACACATAT |
| Q | GAGACATTTTACCTTTTCCGA |
| R | CGTAGTCATTTCTATTTATA |
| S | TCATCGTAGTCATTTCTATTT |
| T | CAACGATGCTTACGTGGCATAT |

In FIG. 5, the sequences of the antisense oligonucleotides represented by each band in the Western blot results of antisense oligonucleotides designed for E0771 cells after transfection with Lipofectamine™ 3000 are shown in the above table.

TABLE 2
Sequence Numbers Corresponding to
the Specific Sequences of Each ASO

| MALT ASO ID | MALT ASO sequence No. |
|---|---|
| A | SEQ ID No. 4 |
| B | SEQ ID No. 5 |
| C | SEQ ID No. 6 |
| D | SEQ ID No. 2 |
| E | SEQ ID No. 3 |
| F | SEQ ID No. 7 |
| G | SEQ ID No. 8 |
| H | SEQ ID No. 9 |
| I | SEQ ID No. 10 |
| J | SEQ ID No. 11 |
| K | SEQ ID No. 12 |
| L | SEQ ID No. 13 |
| M | SEQ ID No. 14 |
| N | SEQ ID No. 15 |
| O | SEQ ID No. 16 |
| P | SEQ ID No. 17 |
| Q | SEQ ID No. 18 |
| R | SEQ ID No. 19 |
| S | SEQ ID No. 20 |
| T | SEQ ID No. 21 |

According to the results, the proteins represented by the sequences in bands B, D, and E show obvious inhibitory results; while the proteins represented by the sequences in bands A, C, G, H, I, J, and K also show good inhibitory results.

Optionally, inhibitory RNA (e.g., dsRNA) is chemically modified to enhance stability or other beneficial properties. Nucleic acids having the characteristics of the present application can be synthesized and/or modified by methods well established in the art.

Optionally, inhibitory nucleic acids may include, for example, siRNA, shRNA, miRNA, and/or miRNA, which are well known in the art and thus will not be described herein.

Those skilled in the art will be able to easily evaluate whether siRNA, shRNA, or miRNA effectively targets the downregulation of the activity of the MALT1 tumor effector region, for example, by transfecting siRNA, shRNA, or miRNA into cells, and detecting the activity of the MALT1 tumor effector region through Western blot (detecting the expression level of the MALT1 tumor effector region) or functional assays (e.g., activation of downstream targets of the MALT1 tumor effector region, such as NF-κB signaling).

Correspondingly, the applications of various substances capable of “inhibiting” the target activity for Invention Point 1 in the treatment of solid tumors are described.

### Example 8

Further experiments were conducted on each mutant strain in Example 4 to explore the mechanism of tumor immune escape, lock the key sites, and determine the mechanism of MALT1 action.

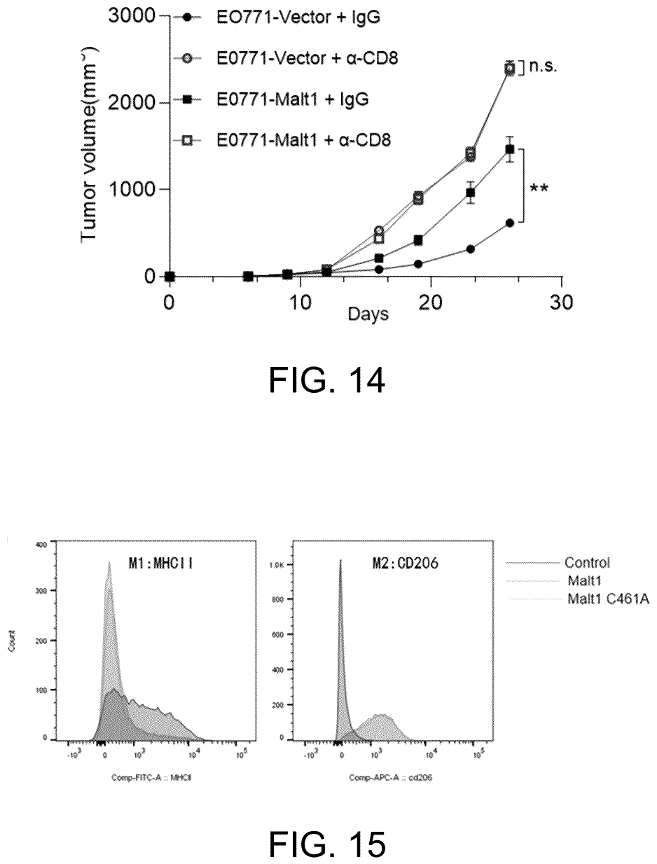

1×10⁵ E0771-VEC and E0771-Malt1 were orthotopically injected into the fat pad of mice with uneliminated (IgG group) or eliminated CD8+ T cells (α-CD8 group). Tumors were measured twice a week. n=12. The experimental results are shown in FIG. 14.

Conditioned media from E0771-VEC, E0771-Malt1, and E0771-Malt1-C461A cells were collected and used to treat bone marrow-derived macrophages (BMDM). After 48 hours, the expression of MHCII and CD206 on BMDM was analyzed by flow cytometry. The flow cytometry experimental results are shown in FIG. 15.

Based on the mutations of each cell line, combined with the corresponding relationship between target-mutated cell lines and their cell numbers shown in FIG. 9, the corresponding relationship between target-mutated cell lines and tumor volumes shown in FIG. 14, and the flow cytometry experimental results verifying the conversion of macrophages from M2-type to M1-type macrophages shown in FIG. 15, it is indicated that: comparison between cell lines 2, 7 and cell line 1 indicates that the effect of MALT1 on macrophage proliferation is independent of Para-caspase activity; comparison results between cell lines 4, 5 and cell line 2, as well as comparison results between cell lines 9, 10 and cell line 7 show that once the discovered key effector target sites V87 and L88 are mutated, the effect of MALT1 on macrophage proliferation disappears immediately.

Therefore, it can be determined that:

The key effector target sites V87 and L88 discovered in the present invention are the key sites for MALT1 to form the CBM complex. Mutation of any key amino acid can disrupt the CBM complex, leading to the complete elimination of the effect of MALT1 on macrophages.

After the above analysis, it was determined that the V87 and L88 sites on the mouse MALT-1 tumor effector region are the key sites for MALT1 to form the CBM complex. The abundance of MALT-1 protein expression in 11 cell lines was compared before and after the addition of macrophages. The results are shown in FIG. 7. The experimental results indicate that after the addition of macrophages, mutations at the V87 and L88 sites significantly reduce the expression of MALT1 protein, as shown in the red box in FIG. 7.

Further, the effect of knocking out the target site on solid tumors was designed:

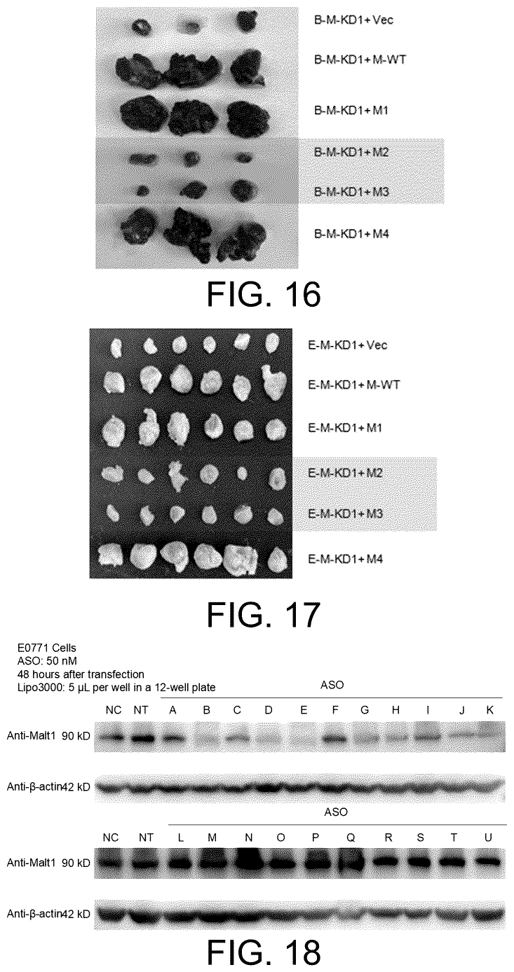

VEC, M-WT, M-WT-C83A, M-WT-V87R, M-WT-L88D, and M-WT-E104R (marked from top to bottom on the right side of FIG. 16) were respectively complemented in B16F10-MALT1-KD cells (melanoma cells) to obtain corresponding cell lines, and then 1×10⁵ corresponding cells were subcutaneously injected into the subcutaneous tissue near the groin of wild-type mice. Tumors were measured twice a week starting from day 6. n=6. FIG. 16 shows the tumor size on day 18.

Right side: VEC, M-WT, M-WT-C83A, M-WT-V87R, M-WT-L88D, and M-WT-E104R (marked from top to bottom on the right side of FIG. 17) were respectively complemented in E0771-MALT1-KD cells (breast cancer cells) to obtain corresponding cell lines, and then 1×10⁵ corresponding cells were orthotopically injected into the fat pad of wild-type mice. Tumors were measured twice a week starting from day 7. n=12. FIG. 17 shows the tumor size on day 19.

It can be seen that mutations at the V87 and L88 sites significantly reduce the tumor volume of melanoma and breast cancer.

### Example 9

After extensive screening, antisense oligonucleotides (ASO) capable of effectively degrading MALT1 have been found. The preparation process and specific antisense oligonucleotide sequences are shown in Example 7.

The antisense oligonucleotide prodrug (ASO24, whose nucleotide sequence is shown in SEQ ID No. 2) can significantly inhibit the further development of cancer cells: the verification effect in breast cancer cells (E0771) is shown in FIG. 18: the prodrug of the antisense oligonucleotide drug has a good effect of inhibiting protein expression. The prodrugs represented by the sequences in bands B, D, and E have obvious inhibitory effects on MALT1 expression, and the prodrugs represented by the sequences in bands A, C, G, H, I, J, and K also show good inhibitory effects on MALT1 expression (the numbers, sequences, and sequence numbers of these bands are shown in Tables 1 and 2).

### Example 10

Two shRNAs (sequences not shown) were used to knock down Malt1 respectively. After knockdown, the cells were very sensitive to CD8 T cell killing, and the cell survival rate decreased significantly. The two shRNAs were used to knock down Malt1 respectively, and the corresponding cell lines were injected subcutaneously into mice. The tumor volume change was monitored, and the growth rate of the knocked-down cells was very slow. The specific experiments are as follows:

### Verification Effect on Melanoma Cells (B16F10)

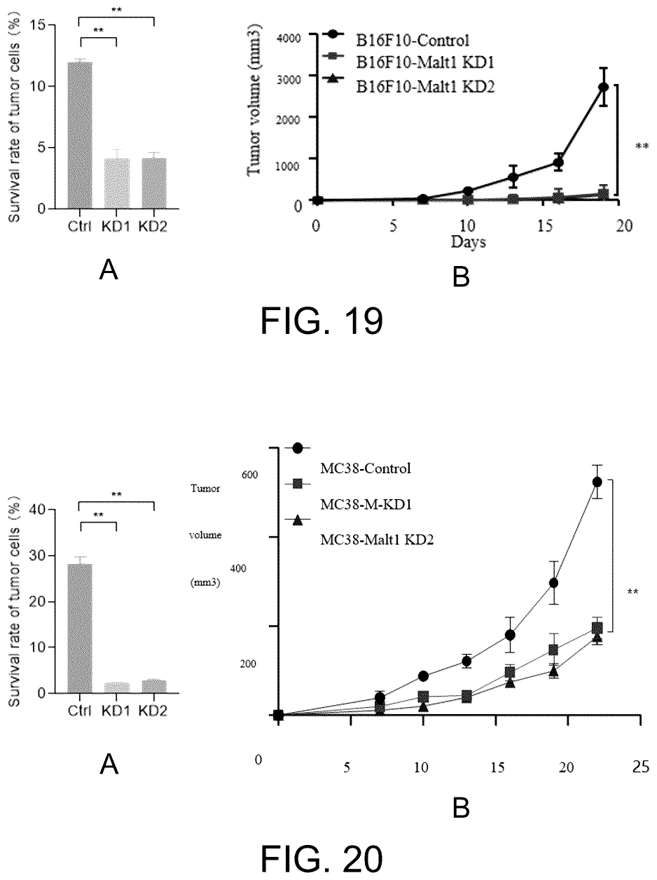

B16F10-CTRL, B16F10-MALT1-KD1, and B16F10-MALT1-KD2 were co-cultured with CD8+ T cells respectively at an effector-to-target ratio (E:T) of 1:1. After 24 hours, luciferase activity was measured to evaluate the survival rate of tumor cells (A of FIG. 19). 1×10⁵ control group (B16F10-CTRL) and MALT1 knockdown group (B16F10-MALT1-KD1 and B16F10-MALT1-KD2) cells were subcutaneously injected into the subcutaneous tissue near the groin of C57BL6/J mice. Tumors were measured starting from day 7, n=14. The tumor volume change curve results are shown in B of FIG. 19.

### Verification Effect on Colon Cancer Cells (MC38)

MC38-CTRL, MC38-MALT1-KD1, and MC38-MALT1-KD2 were co-cultured with CD8+ T cells respectively at an E:T ratio of 1:1. After 24 hours, luciferase activity was measured to evaluate the survival rate of tumor cells (A of FIG. 20). Right side: 1×10⁵ control group (MC38-CTRL) and MALT1 knockdown group (MC38-MALT1-KD1 and MC38-MALT1-KD2) cells were subcutaneously injected into the subcutaneous tissue near the groin of C57BL6/J mice. Tumors were measured starting from day 7, n=14. The tumor volume change curve results are shown in B of FIG. 20.

### Example 11

Comparison of the effects of ASOs of the present invention (ASO24 and ASO25, whose nucleotide sequences are shown in SEQ ID No. 2 and 3 respectively) with existing MALT1-targeted drugs.

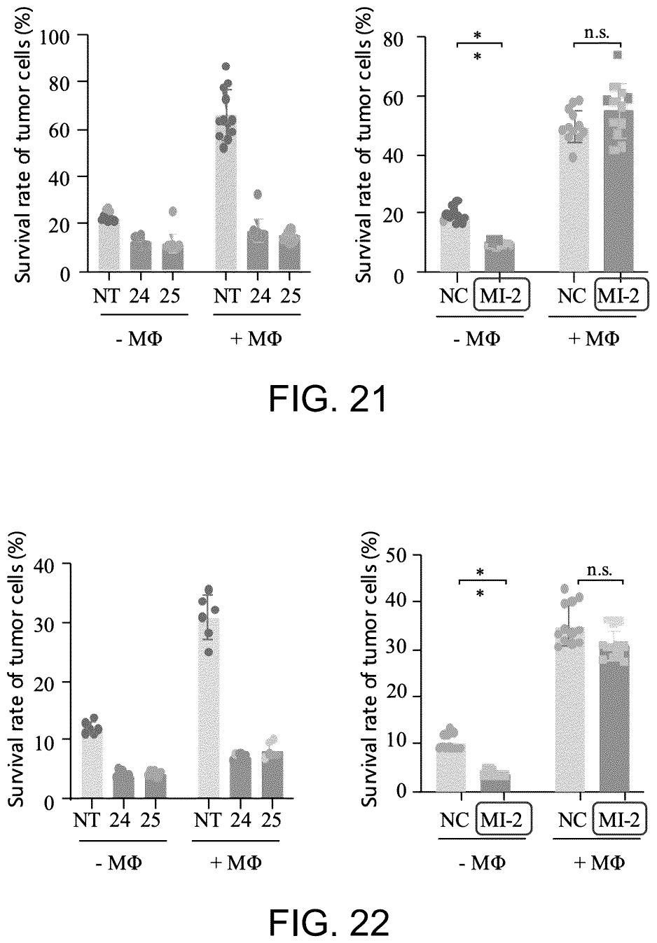

The verification results of the survival rate of mouse breast cancer cells (E0771) are shown in A and B of FIG. 21. Survival rates of E0771 cells treated with ASO NT (nontarget control), ASO 24, or ASO 25 (nucleotide sequences shown in SEQ ID NO. 2 and 3 respectively) in the CD8+ T cell killing system (−MΦ) and the three-cell co-culture system (+MΦ). The ratio of tumor cells:macrophages:CD8+ T cells was 1:2:2. n=12. Data are expressed as mean±SD. Statistical analysis was performed using Student's t test. ** represents p<0.01. Survival rates of E0771 cells treated with DMSO or MALT1 protease inhibitor (MI-2) at a final concentration of 10 μg/mL in the CD8+ T cell killing system (−MΦ) and the three-cell co-culture system (+MΦ). The ratio of tumor cells:macrophages:CD8+ T cells was 1:2:2. n=5. Data are expressed as mean±SD. Statistical analysis was performed using Student's t test. n.s. represents no significant difference. ** represents p<0.01. In the figure: NC: control; MI-2: MALT1-targeted drug targeting para-caspase; NT: control not targeting any sequence of the mouse genome; 24, 25: effective ASO prodrugs administered. Among them, MI-2 (Histone Methyltransferase inhibitor) is an existing MALT1-targeted drug that inhibits Menin-MLL interaction and is an irreversible MALT1 inhibitor. Its structural formula is as follows:

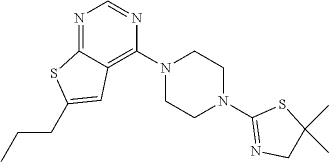 [SMILES: CCCc1cc2c(N3CCN(C4=NCC(C)(C)S4)CC3)ncnc2s1]

The verification results of the survival rate of mouse melanoma cells (B16F10) are shown in A and B of FIG. 22. Survival rates of B16F10 cells treated with ASO NT, ASO24, or ASO25 in the CD8+ T cell killing system (−MΦ) and the three-cell co-culture system (+MΦ). The ratio of tumor cells:macrophages:CD8+ T cells was 1:2:2. n=6. Data are expressed as mean±SD. Statistical analysis was performed using Student's t test. ** represents p<0.01. (d) Survival rates of B16F10 cells treated with DMSO or MALT1 protease inhibitor (MI-2) at a final concentration of 10 μg/mL in the CD8+ T cell killing system (−MΦ) and the three-cell co-culture system (+MΦ). The ratio of tumor cells:macrophages:CD8+ T cells was 1:2:2. n=5. Data are expressed as mean±SD. Statistical analysis was performed using Student's t test. n.s. represents no significant difference. ** represents p<0.01. In the figure: NC: control; MI-2: MALT1-targeted drug targeting para-caspase; NT: control not targeting any sequence of the mouse genome; 24, 25: effective ASO prodrugs administered.

Therefore, the lower survival rate of cancer cells proves that the ASO drug of the present invention has a more significant effect, which is superior to existing MALT1-targeted drugs. Compared with MI-2, the antisense oligonucleotide prodrug has a lower tumor survival rate and more significant effect.

### Example 12

Based on the intratumoral experimental data of various mouse breast cancer models and melanoma models (administration started 7 days later, once every 3 days), this example studies the effect comparison between the ASO prodrug (ASO24, whose sequence is shown in SEQ ID No. 2) and PD-1/PD-L1, as well as the effect of their combination.

1×10⁵ E0771 cells were orthotopically injected into the fat pad of wild-type mice. Starting from day 7, PD-1 antibody (BioXCell #BE0033, clone J43, the same below) monotherapy, ASO monotherapy, or combined PD-1 antibody and ASO therapy was initiated. For anti-PD-1 antibody monotherapy, intraperitoneal injection was performed once a week at a dose of 3 mg/kg starting 7 days after tumor transplantation, and the control group was injected with DPBS. For MALT1 ASO monotherapy, in vivo-jetPEI®, in vivo DNA transfection kit was used according to the instructions. Intratumoral injection was performed twice a week at a dose of 5 nmol starting 7 days after tumor transplantation, and the control group was injected with solvent or nontarget ASO. Combined therapy will receive the two specified monotherapies. Tumor measurement was started after administration, once every three days. n=12. Data are expressed as mean. Statistical analysis was performed using Two way ANOVA. ** represents p <0.01. The results are shown in Table 3.

TABLE 3
Comparison of Efficacy Between Anti-PD-1 Antibody and ASO Prodrug,
and Combined Therapy for Breast Cancer (E0771 Cells)

| | | | | anti-pd1 + |
|---|---|---|---|---|
| | Placebo | anti-pd1 | ASO24 | ASO24 |
| Day | Group (mm³) | Group (mm³) | Group (mm³) | Group (mm³) |
| 0 | 0 | 0 | 0 | 0 |
| 7 | 45 | 46 | 49 | 51 |
| 10 | 88 | 56 | 36 | 13 |
| 13 | 180 | 86 | 92 | 13 |
| 16 | 340 | 162 | 140 | 13 |
| 19 | 916 | 321 | 306 | 13 |

Note:
Tumor volume was measured by electronic vernier caliper for length and width. Estimated volume = 0.5 × length × length × width.

1×10⁵ B16F10 cells were subcutaneously injected near the subcutaneous tissue of the groin of wild-type mice. Starting from day 7, PD-1 antibody monotherapy, ASO monotherapy, or combined PD-1 antibody and ASO therapy was initiated. n=10. Data are expressed as mean. Statistical analysis was performed using Two way ANOVA. ** represents p<0.01. The results are shown in Table 4.

TABLE 4
Comparison of Efficacy Between Anti-PD-1 Antibody
and ASO Precursor Drugs and Their Combined
Therapy for Melanoma (B16F10 Cells)

| | | | | anti-pd1 + |
|---|---|---|---|---|
| | Placebo | anti-pd1 | ASO24 | ASO24 |
| Day | Group (mm³) | Group (mm³) | Group (mm³) | Group (mm³) |
| 0 | 0 | 0 | 0 | 0 |
| 7 | 43 | 42 | 49 | 44 |
| 10 | 138 | 52 | 26 | 13 |
| 13 | 401 | 114 | 129 | 13 |
| 16 | 714 | 249 | 311 | 13 |

Note:
Tumor volume was measured by electronic vernier caliper for length and width. Estimated volume = 0.5 × length × length × width.

1×10⁵ PyMT-A cells were orthotopically injected into the fat pad of wild-type mice. Starting from day 7, PD-1 antibody monotherapy, ASO monotherapy, or combined PD-1 antibody and ASO therapy was initiated. n=10. Data are expressed as mean. Statistical analysis was performed using Two way ANOVA. n.s. represents no significant difference. ** represents p <0.01. The results are shown in Table 5.

TABLE 5
Comparison of Efficacy Between Anti-PD-1 Antibody
and ASO Precursor Drugs and Their Combined
Therapy for Breast Cancer (PyMT-A Cells)

| | | | | anti-pd1 + |
|---|---|---|---|---|
| | Placebo | anti-pd1 | ASO24 | ASO24 |
| Day | Group (mm³) | Group (mm³) | Group (mm³) | Group (mm³) |
| 0 | 0 | 0 | 0 | 0 |
| 7 | 13 | 13 | 13 | 13 |
| 10 | 78 | 50 | 41 | 18 |
| 13 | 244 | 169 | 142 | 67 |
| 16 | 389 | 282 | 245 | 81 |
| 19 | 996 | 947 | 578 | 146 |

Note:
Tumor volume was measured by electronic vernier caliper for length and width. Estimated volume = 0.5 × length × length × width.

2×10⁵ 4T1 cells were orthotopically injected into the fat pad of wild-type mice. Starting from day 7, PD-1 antibody monotherapy, ASO monotherapy, or combined PD-1 antibody and ASO therapy was initiated. n=10. Data are expressed as mean. Statistical analysis was performed using Two way ANOVA. n.s. represents no significant difference. ** represents p <0.01. The results are shown in Table 6.

TABLE 6
Comparison of Efficacy Between Anti-PD-1 Antibody
and ASO Precursor Drugs and Their Combined Treatment
of Breast Cancer Cells (4T1 Cells)

| | | | | anti-pd1 + |
|---|---|---|---|---|
| | Placebo | anti-pd1 | ASO24 | ASO24 |
| Day | Group (mm³) | Group (mm³) | Group (mm³) | Group (mm³) |
| 0 | 0 | 0 | 0 | 0 |
| 7 | 64 | 67 | 66 | 70 |
| 10 | 147 | 159 | 71 | 67 |
| 13 | 387 | 407 | 205 | 133 |
| 16 | 625 | 584 | 377 | 240 |
| 19 | 1358 | 1347 | 876 | 306 |

Note:
Tumor volume was measured by electronic vernier caliper for length and width. Estimated volume = 0.5 × length × length × width.

It can be seen from the data in Tables 3-6: PD1 drugs have almost no effect in PyMT-A and 4T1 breast cancers, but ASO drugs have significant effects. Overall, ASO drugs perform better than or equivalent to PD1 drugs. The combined use of ASO drugs and PD1 drugs shows excellent effects in all four models, and ASO drugs and PD1 drugs produce a synergistic effect.

Study on the Effect Comparison between ASO Prodrug and PD-1/PD-L1 and Their Combined Use Based on Systemic Administration Experimental Data of Various Mouse Breast Cancer Models and Melanoma Models (Administration Started 7 Days Later, Once Every 3 Days).

The experimental process is similar to the intratumoral administration of ASO24 in E0771 cells, with the difference that ASO24 is administered via systemic delivery by tail vein injection of LNP-ASO in mice.

TABLE 7
Comparison of the effects of anti-PD-1 antibody and
ASO prodrug via systemic administration and their
combined therapy for breast cancer (E0771 cells)

| | | | | anti-pd1 + |
|---|---|---|---|---|
| | Placebo | anti-pd1 | ASO24 | ASO24 |
| Day | Group (mm³) | Group (mm³) | Group (mm³) | Group (mm³) |
| 0 | 0 | 0 | 0 | 0 |
| 7 | 30 | 30 | 29 | 30 |
| 9 | 74 | 42 | 51 | 28 |
| 11 | 107 | 61 | 57 | 25 |
| 13 | 215 | 110 | 86 | 43 |
| 15 | 378 | 188 | 195 | 53 |
| 17 | 540 | 228 | 225 | 49 |
| 19 | 819 | 397 | 313 | 88 |

Note:
Tumor volume was measured by electronic vernier caliper for length and width. Estimated volume = 0.5 × length × length × width.

TABLE 8
Comparison of the effects of anti-PD-1 antibody and
ASO prodrug via systemic administration and their
combined therapy for breast cancer cells (4T1 cells)

| | | | | anti-pd1 + |
|---|---|---|---|---|
| | Placebo | anti-pd1 | ASO24 | ASO24 |
| Day | Group (mm³) | Group (mm³) | Group (mm³) | Group (mm³) |
| 0 | 0 | 0 | 0 | 0 |
| 7 | 41 | 42 | 44 | 43 |
| 9 | 112 | 98 | 94 | 71 |
| 11 | 172 | 158 | 103 | 71 |
| 13 | 223 | 222 | 136 | 99 |
| 15 | 413 | 400 | 244 | 168 |
| 17 | 646 | 597 | 333 | 190 |
| 19 | 831 | 861 | 510 | 318 |

Note:
Tumor volume was measured by electronic vernier caliper for length and width. Estimated volume = 0.5 × length × length × width.

It can be seen from the data in Tables 7-8: via systemic administration of ASO24, PD1 drugs have no effect in 4T1 breast cancer, but ASO drugs have significant effects. ASO drugs perform better than PD1 drugs. The combined use of ASO drugs and PD1 drugs shows excellent effects in both models, that is, ASO drugs and PD1 drugs produce a synergistic effect.

### Example 13

To verify the therapeutic effect of ASO (ASO24, whose sequence is shown in SEQ ID No. 2) on other solid cancer types besides breast cancer and melanoma, animal models of lung cancer, liver cancer, and colorectal cancer were used to verify the combination of ASO and PD-1/PD-L1.

### Therapeutic Regimen of Combined PD-1 Antibody and ASO in Mouse Lung Cancer Model:

Wild-type C57BL/6J mice were inoculated with 10 μl of lung cancer cell mixture (containing 1×10⁶ LLC cells/mouse) near the groin. Treatment was initiated on day 7 after inoculation. PD-1 antibody (BioXCell #BE0033, clone J43) was administered intraperitoneally once a week at a dose of 3 mg/kg. LNP-ASO was administered via tail vein injection every other day at a dose of 50 μg. Experimental mice were sacrificed on day 15 after inoculation. There were 5 mice in each experimental group. Tumor size was measured, and the average tumor volume of each group of mice was calculated. In addition, controls with only PD-1 antibody or LNP-ASO administration and blank controls were set. The administration procedure and dosage were the same as those of the combined use.

### Therapeutic Regimen of Combined PD-1 Antibody and ASO in Mouse Liver Cancer Model:

Wild-type BALB/c mice were subcutaneously inoculated with Hep53.4 liver cancer cells near the groin at a cell inoculation dose of 1×10⁶ cells/mouse. Treatment was initiated on day 7 after inoculation. PD-1 antibody (BioXCell #BE0033, clone J43) was administered intraperitoneally once a week at a dose of 3 mg/kg. LNP-ASO was administered via tail vein injection every other day at a dose of 50 μg. Experimental mice were sacrificed on day 15 after inoculation. There were 5 mice in each experimental group. Tumor size was measured, and the average tumor volume of each group of mice was calculated. In addition, controls with only PD-1 antibody or LNP-ASO administration and blank controls were set.

### Therapeutic Regimen of Combined PD-1 Antibody and ASO in Mouse Colorectal Cancer Model:

BALB/c mice were subcutaneously inoculated with CT26 colorectal cancer cells at a cell inoculation dose of 1×10⁶ cells/mouse. After 3 weeks, the mice were euthanized, and the tumor tissue blocks were cut into 1 mm×1 mm×1 mm. The mice were anesthetized, and the tissue blocks were orthotopically transplanted (sutured to the damaged serosal tissue of the cecum or directly inserted into the serosa). Treatment was initiated on week 2 after transplantation. PD-1 antibody (BioXCell #BE0033, clone J43) was administered intraperitoneally once a week at a dose of 3 mg/kg. LNP-ASO was administered via tail vein injection every other day at a dose of 50 μg. Experimental mice were sacrificed on day 15 after transplantation. There were 5 mice in each experimental group. Tumor size was measured, and the average tumor volume of each group of mice was calculated. In addition, controls with only PD-1 antibody or LNP-ASO administration and blank controls were set. The administration procedure and dosage were the same as those of the combined use.

### Therapeutic Regimen of Combined PD-L1 Antibody and ASO in Mouse Lung Cancer Model:

Wild-type C57BL/6J mice were inoculated with 10 μl of lung cancer cell mixture (containing 1×10⁶ LLC cells/mouse) near the groin. Treatment was initiated on day 7 after inoculation. PD-L1 antibody (BioXCell #: BE0101; Clone: 10F.9G2) was administered intraperitoneally once a week at a dose of 10 mg/kg. LNP-ASO was administered via tail vein injection every other day at a dose of 50 μg. Experimental mice were sacrificed on day 15 after inoculation. There were 5 mice in each experimental group. Tumor size was measured, and the average tumor volume of each group of mice was calculated. In addition, controls with only PD-L1 antibody or LNP-ASO administration and blank controls were set. The administration procedure and dosage were the same as those of the combined use.

### Therapeutic Regimen of Combined PD-L1 Antibody and ASO in Mouse Liver Cancer Model:

Wild-type BALB/c mice were subcutaneously inoculated with Hep53.4 liver cancer cells near the groin at a cell inoculation dose of 1×10⁶ cells/mouse. Treatment was initiated on day 7 after inoculation. PD-L1 antibody (BioXCell #: BE0101; Clone: 10F.9G2) was administered intraperitoneally once a week at a dose of 10 mg/kg. LNP-ASO was administered via tail vein injection every other day at a dose of 50 μg. Experimental mice were sacrificed on day 15 after inoculation. There were 5 mice in each experimental group. Tumor size was measured, and the average tumor volume of each group of mice was calculated. In addition, controls with only PD-L1 antibody or LNP-ASO administration and blank controls were set.

### Therapeutic Regimen of Combined PD-L1 Antibody and ASO in Mouse Colorectal Cancer Model:

BALB/c mice were subcutaneously inoculated with CT26 colorectal cancer cells at a cell inoculation dose of 1×10⁶ cells/mouse. After 3 weeks, the mice were euthanized, and the tumor tissue blocks were cut into 1 mm×1 mm×1 mm. The mice were anesthetized, and the tissue blocks were orthotopically transplanted (sutured to the damaged serosal tissue of the cecum or directly inserted into the serosa). Treatment was initiated on week 2 after transplantation. PD-L1 antibody (BioXCell #: BE0101; Clone: 10F.9G2) was administered intraperitoneally once a week at a dose of 10 mg/kg. LNP-ASO was administered via tail vein injection every other day at a dose of 50 μg. Experimental mice were sacrificed on day 15 after transplantation. There were 5 mice in each experimental group. Tumor size was measured, and the average tumor volume of each group of mice was calculated. In addition, controls with only PD-L1 antibody or LNP-ASO administration and blank controls were set. The administration procedure and dosage were the same as those of the combined use.

The experimental results are summarized in Table 9.

TABLE 9
Therapeutic verification results of combined LNP-ASO with PD-1 antibody and PD-L1
antibody in animal models of lung cancer, liver cancer, and colorectal cancer

| | | | | | Combined | Combined | |
|---|---|---|---|---|---|---|---|
| | | PD-1 | PD-L1 | | PD-1 | PD-L1 | |
| | Blank | antibody | antibody | LNP-ASO | antibody and | antibody and | |
| Animal | control | alone | alone | alone | LNP-ASO | LNP-ASO | Experimental |
| model | (mm³) | (mm³) | (mm³) | (mm³) | (mm³) | (mm³) | duration |
| Lung | 24 | 24 | 24 | 24 | 24 | 24 | Day 7 |
| cancer | 68 | 50 | 52 | 38 | 18 | 20 | Day 9 |
| LLC | 140 | 123 | 124 | 59 | 16 | 19 | Day 11 |
| cells - | 232 | 185 | 180 | 112 | 18 | 15 | Day 13 |
| C57BL/ | 413 | 376 | 383 | 197 | 20 | 12 | Day 15 |
| 6J mice | | | | | | | |
| Hep53.4 | 22 | 22 | 22 | 22 | 22 | 22 | Day 7 |
| liver cancer | 25 | 24 | 25 | 24 | 22 | 22 | Day 9 |
| cells - | 36 | 34 | 33 | 28 | 25 | 24 | Day 11 |
| BALB/c | 48 | 47 | 46 | 35 | 28 | 27 | Day 13 |
| mice | 60 | 62 | 59 | 44 | 32 | 30 | Day 15 |
| CT26 | 28 | 28 | 28 | 28 | 28 | 28 | Day 7 |
| colorectal | 62 | 38 | 40 | 36 | 28 | 29 | Day 9 |
| cancer | 116 | 59 | 62 | 67 | 20 | 18 | Day 11 |
| cells - | 205 | 130 | 133 | 120 | 6 | 5 | Day 13 |
| BALB/c | 421 | 254 | 260 | 210 | 6 | 5 | Day 15 |
| mice | | | | | | | |

Note:
Tumor volume was measured by electronic vernier caliper for length and width. Estimated volume = 0.5 × length × length × width.

It can be seen from the data in Table 9 that the ASO prodrug of the present invention has therapeutic effects on various solid cancer model animals. Compared with PD-1 antibody/PD-L1 antibody, its therapeutic effect is equivalent or better. When combined with PD-1 antibody/PD-L1 antibody, the therapeutic effect is excellent, showing a significant synergistic effect.

The above are only preferred embodiments of the present invention and are not intended to limit the present invention. Although the present invention has been disclosed in the preferred embodiments as above, it is not intended to limit the present invention. Any person skilled in the art can make some modifications or changes to equivalent embodiments without departing from the scope of the technical solution of the present invention. However, any simple modifications, equivalent changes, and modifications made to the above embodiments according to the technical essence of the present invention without departing from the content of the technical solution of the present invention all fall within the scope of the technical solution of the present invention.

1. A MALT1 tumor inhibitor, wherein it can inhibit the expression of the MALT1 gene or inactivate the function of the MALT1 protein;
the MALT1 tumor inhibitor is selected from agents that can affect the expression of the death domain of the MALT1 protein, agents that knockout the death domain, agents that alter the structure of the death domain, or agents that degrade the death domain.

2. The MALT1 tumor inhibitor according to claim 1, wherein it targets the protein-protein interaction site between MALT1 and BCL-10, thereby reducing or abolishing the binding ability between MALT1 and BCL-10.

3. The MALT1 tumor inhibitor according to claim 2, wherein it can also inhibit MALT1 enzymatic activity.

4. The MALT1 tumor inhibitor according to claim 1, wherein it acts on at least amino acid residues 64 to 114 based on the MALT1 amino acid sequence shown in SEQ ID No. 1, or the nucleotide sequence encoding the same.

5. The MALT1 tumor inhibitor according to claim 4, wherein it acts on at least amino acid residue 81 and/or 82 based on the MALT1 amino acid sequence shown in SEQ ID No. 1, or the corresponding codon on the nucleotide sequence encoding the same.

6. The MALT1 tumor inhibitor according to claim 1, wherein it comprises an agent that inhibits, blocks, or disrupts the protein-protein interaction between MALT1 and BCL10.

7. The MALT1 tumor inhibitor according to claim 6, wherein the agent that inhibits, blocks, or disrupts the protein-protein interaction between MALT1 and BCL10 is selected from covalent cross-linking agents that bind to the MALT1 death domain, polyclonal antibodies against the MALT1 death domain, monoclonal antibodies against the MALT1 death domain, small molecule inhibitors that inhibit the protein-protein interaction between MALT1 and BCL10, reagents for point mutation of the binding site between the MALT1 death domain and BCL10, or reagents for eliminating the binding site between the MALT1 death domain and BCL10 through homologous recombination or gene editing;
preferably, the covalent cross-linking agents that bind to the MALT1 death domain are selected from antioxidants, non-steroidal anti-inflammatory drugs (NSAIDs), natural compounds, proteasome inhibitors, IKK inhibitors, NF-κB-specific antisense oligonucleotides, or cell-penetrating peptides;
preferably, the antioxidants comprise alantolactone and N-acetylcysteine (NAC);
the non-steroidal anti-inflammatory drugs (NSAIDs) comprise cyclooxygenase-2 (COX-2);
the natural compounds comprise cantharidin, curcumin, ursolic acid, and resveratrol;
the proteasome inhibitors comprise proteasome inhibitor PS341 and melatonin;
the IKK inhibitor comprises the tumor suppressor protein phosphatase PHLPP2;
the cell-penetrating peptides comprise the cell-penetrating peptide inhibitor SN50;
preferably, the mutagens for the protein-protein interaction site between MALT1 and BCL10 comprise: reagents for point mutation of amino acid residue 81 and/or 82 of the MALT1 death domain, CRISPR-Cas9 gene cleavage reagents, and reagents for homologous recombination of the protein-protein interaction site between MALT1 and BCL10.

8. The MALT1 tumor inhibitor according to claim 1, wherein it alters the tumor tissue microenvironment, significantly reducing the number of macrophages in the tumor tissue and converting macrophages in the tumor tissue from M2 type to M1 type.

9. The MALT1 tumor inhibitor according to claim 1, wherein it is an antisense oligonucleotide that inactivates MALT1.

10. The MALT1 tumor inhibitor according to claim 9, wherein the nucleotide sequence of the antisense oligonucleotide is as shown in any one of SEQ ID No. 2-6 and 8-12.

11. The MALT1 tumor inhibitor according to claim 10, wherein the nucleotide sequence of the antisense oligonucleotide is as shown in any one of SEQ ID No. 2-3.

12. The MALT1 tumor inhibitor according to claim 9, wherein the antisense oligonucleotide is chemically modified;
preferably, the antisense oligonucleotide further comprises modified internucleoside linkages;
preferably, the antisense oligonucleotide further comprises modified sugars;
preferably, the antisense oligonucleotide further comprises modified nucleobases.

13. The MALT1 tumor inhibitor according to claim 9, wherein the antisense oligonucleotide is delivered by a drug delivery system.

14. The MALT1 tumor inhibitor according to claim 1, wherein the agent for degrading the MALT1 death domain is selected from reagents of PROTAC, molecular glue, LYTAC, MODE, ATAC, Apt-LYTAC, AbTAC, PROTAB, REULR, KineTAC, IFLD, ATTEC, AUTAC, or AUTOTAC pathways.

15. A pharmaceutical composition for treating tumors, wherein the pharmaceutical composition comprises an effective amount of the MALT1 tumor inhibitor according to claim 1, and a pharmaceutically acceptable carrier.

16. The pharmaceutical composition according to claim 15, wherein it further comprises a second drug;
the second drug is selected from immune checkpoint inhibitors, chemotherapeutic agents or MALT1 enzymatic activity inhibitors;
the MALT1 enzymatic activity inhibitor is a small-molecule inhibitor.

17. (canceled)

18. The pharmaceutical composition according to claim 16, wherein the immune checkpoint inhibitor is selected from PD-L1, PD-L2, PD-1, CTLA-4, TIM-3, LAG-3, VISTA, or TIGIT inhibitors.

19. The pharmaceutical composition according to claim 16, wherein the chemotherapeutic agent is selected from alkylating agents, antimetabolites, antitumor antibiotics, plant-derived agents, hormonal agents, and other types of chemotherapeutic agents;
preferably:
the alkylating agents comprise nimustine, carmustine, lomustine, cyclophosphamide, ifosfamide, or glyfosfin;
the antimetabolites comprise doxifluridine, doxifluridine, 5-fluorouracil, mercaptopurine, thioguanine, fludarabine, tegafur, gemcitabine, ftorafur, hydroxyurea, methotrexate, uftoral, or ancitabine;
the antitumor antibiotics comprise dactinomycin D, doxorubicin, daunorubicin, epirubicin, mitomycin, bleomycin, or pirarubicin;
the antitumor plant and animal-derived agents comprise irinotecan, harringtonine, hydroxycamptothecin, vinorelbine, paclitaxel, docetaxel, topotecan, vincristine, vindesine, vinblastine, teniposide, etoposide, or elemene;
the antitumor hormonal agents comprise exemestane, anastrozole, aminoglutethimide, letrozole, formestane, medroxyprogesterone acetate, tamoxifen, or toremifene;
the other types of chemotherapeutic agents comprise asparaginase, carboplatin, cisplatin, dacarbazine, oxaliplatin, eloxatin, lobaplatin, mitoxantrone, or procarbazine.

20. (canceled)

21. (canceled)

22. (canceled)

23. (canceled)

24. Use of the MALT1 tumor inhibitor according to claim 1 in the treatment of diseases, wherein the diseases comprise tumors.

25. The use according to claim 24, wherein the applicable disease is solid tumors;
the solid tumors comprise but are not limited to malignant tumors occurring in the lungs, pancreas, liver, digestive tract, reproductive system and other parts, such as Hodgkin's lymphoma, non-Hodgkin's lymphoma, lung cancer, breast cancer (comprising triple-negative breast cancer), melanoma, ovarian cancer, gastric cancer, colon cancer, rectal cancer, liver cancer, pancreatic cancer, head and neck malignant tumors, urinary system malignant tumors, endometrial cancer, cervical cancer, osteosarcoma, chondrosarcoma, Ewing's sarcoma, thyroid cancer, hepatoblastoma, and nephroblastoma;
wherein the applicable subject is a mammal;
wherein the applicable subject is a human.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
